(19) United States
(12) Patent Application Publication
(10) Pub. No.: US 2010/0279919 A1
(43) Pub. Date: Nov. 4, 2010

(54) COMPOSITIONS COMPRISING HUMAN INTEGRIN-LINKED KINASE-SIRNA AND METHODS OF USE THEREOF

(75) Inventor: Frank Y. Xie, Germantown, MD (US)

Correspondence Address: SEED INTELLECTUAL PROPERTY LAW GROUP PLLC, 701 FIFTH AVE, SUITE 5400, SEATTLE, WA, 98104, US

(73) Assignee: INTRADIGM CORPORATION, Palo Alto, CA (US)

(21) Appl. No.: 12/676,504

(22) PCT Filed: Sep. 4, 2008
(86) PCT No.: PCT/US08/75264

§ 371 Date: Jun. 21, 2010

Related U.S. Application Data
(60) Provisional application No. 60/969,880, filed on Sep. 4, 2007.

(51) Int. Cl. A61K 38/02; C07H 21/02; A61K 31/713; C12N 5/10; A61P 35/00; A61P 11/06; A61P 17/06; A61P 3/06; A61P 19/02; C12N 15/63; C12N 5/02

(52) U.S. Cl. 514/1.1; 536/24.5; 514/44 A; 435/325; 435/320.1; 435/375

(57) ABSTRACT

The present invention provides nucleic acid molecules that inhibit ILK expression. Methods of using the nucleic acid molecules are also provided.

### CROSS-REFERENCE TO RELATED APPLICATION

This application claims the benefit under 35 U.S.C. §119(e) of U.S. Provisional Patent Application No. 60/969,880 filed Sep. 4, 2007, which is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480251_{—}402PC_SEQUENCE_LISTING.txt. The text file is 54 KB, was created on Sep. 4, 2008, and is being submitted electronically via EFS-Web, concurrent with the filing of the specification.

### BACKGROUND OF THE INVENTION

1. Field of the Invention

The present invention relates to siRNA molecules for modulating the expression of human integrin-linked kinase (ILK) and the application of these siRNA molecules as therapeutic agents for human diseases including a variety of cancers, metabolic disorders, inflammatory diseases and diseases associated with abnormal or pathological angiogenesis.

2. Description of the Related Art

The primary contacts made between cells and the surrounding environment, including contacts with other cells, are mediated by the transmembrane proteins known as integrins. These contacts are critical for the bidirectional transduction of signals to the interior of the cell and consequently to the modulation of biochemical pathways. Integrins are heterodimeric cation-dependent membrane-spanning glycoproteins that mediate cell adhesion, migration and signal transduction. Integrins are composed of an alpha and beta subunit and to date, 8 beta and 15 alpha subunits have been identified which combine to form over 20 different αβ heterodimers. Integrins have been found in all tissues examined and consist of a large extracellular domain, a transmembrane domain and a smaller cytoplasmic domain. It is the extracellular domain of the integrin that acts as a receptor for various matrix proteins, while the cytoplasmic domain has been shown to interact with actin filaments of the cytoskeleton and with cytoplasmic proteins such as talin, paxillin, filamin and focal adhesion kinase (FAK) (LaFlamme et al., Matrix Biol., 1997, 16, 153-163). Recently, four additional proteins that interact with β-integrin subunit cytoplasmic domains were reported. Of the four, only the ankyrin repeat containing serine/threonine protein integrin-linked kinase (ILK) has been shown to bind multiple forms of the integrin beta subunit (Dedhar and Hannigan, Curr. Opin. Cell. Biol., 1996, 8, 657-669; Hannigan et al., Nature, 1996, 379, 91-96).

Integrin-linked kinase (also known as ILK and p59ILK) was originally identified from a two-hybrid screen of a human placental cDNA library by its ability to bind to and phosphorylate the β1-integrin cytoplasmic domain (Hannigan et al., Nature, 1996, 379, 91-96). Characterization of integrin-linked kinase in these studies also revealed that overexpression leads to disrupted epithelial morphology of IEC-18 cells, decreased cell adhesion to extracellular matrix substrates as well as anchorage-independent growth (Hannigan et al., Nature, 1996, 379, 91-96). Others have shown that overexpression of integrin-linked kinase leads to stimulation of the cell cycle, fibronectin matrix assembly, reduced expression of E-cadherin and malignant transformation (Radeva et al., J. Biol. Chem., 1997, 272, 13937-13944; Wu et al., J. Biol. Chem., 1998, 273, 528-536). Interestingly, the integrin-linked kinase gene, which maps to chromosome 11 p15.5, is located in a region associated with genomic imprinting, whereby the expression level of the alleles of a gene depends upon their parental origin and loss of heterozygosity in certain tumor types (Hannigan et al., Genomics, 1997, 42, 177-179).

The expression pattern of integrin-linked kinase is distributed among most human tissues and has been shown to be overexpressed in certain tumors, those being Ewing's sarcoma, primitive neuroectodermal tumor (PNET), medulloblastoma and neuroblastoma (Chung et al., Virchows Arch., 1998, 433, 113-117). Recently it was demonstrated that integrin-linked kinase expression is regulated by erbB-2, a member of the epidermal growth factor receptor family, which plays a pivotal role in epidermal growth and differentiation. The investigators showed that overexpression of erbB-2 led to a specific increase in integrin-linked kinase expression in several regions of epidermal tissue (Xie et al., Am. J. Pathol., 1998, 153, 367-372). These studies implicate integrin-linked kinase in skin development and the pathogenesis of skin diseases.

Integrin-linked kinase also triggers the LEF-1/beta catenin signaling pathway when overexpressed, indicating a role in the activation of transcription within the Wnt signaling cascade (Novak et al., Proc. Natl. Acad. Sci. U.S.A., 1998, 95, 4374-4379). The activity of integrin-linked kinase has been shown to be modulated within other signaling pathways including those involving G-proteins (Tu et al., Mol. Cell. Biol., 1999, 19, 2425-2434) phosphotidylinositol 3-kinase, protein kinase B and glycogen synthase kinase 3 (Delcommenne et al., Proc. Natl. Acad. Sci. U.S.A., 1998, 95, 11211-11216). These results indicate that integrin-linked kinase may play a role in insulin-dependent responses in the cell and possible in the development of diabetes.

Currently, there are no known therapeutic agents which effectively inhibit the synthesis of integrin-linked kinase. Consequently, there remains a long felt need for agents capable of effectively inhibiting integrin-linked kinase function.

RNAi technology is emerging as an effective means for reducing the expression of specific gene products and may therefore prove to be uniquely useful in a number of therapeutic, diagnostic, and research applications for the modulation of expression of ILK. The present invention provides compositions and methods for modulating expression of these proteins using RNAi technology.

The following is a discussion of relevant art pertaining to RNAi. The discussion is provided only for understanding of the invention that follows. The summary is not an admission that any of the work described below is prior art to the claimed invention.

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). The corresponding process in plants (Heifetz et al., International PCT Publication No. WO 99/61631) is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Bahramian and Zarbl, 1999, Molecular and Cellular Biology, 19, 274-283 and Wianny and Goetz, 1999, Nature Cell Biol., 2, 70, describe RNAi mediated by dsRNA in mammalian systems. Hammond et al., 2000, Nature, 404, 293, describe RNAi in Drosophila cells transfected with dsRNA. Elbashir et al., 2001, Nature, 411, 494 and Tuschl et al., International PCT Publication No. WO 01/75164, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877 and Tuschl et al., International PCT Publication No. WO 01/75164) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J, 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two-nucleotide 3′-overhangs with deoxyribonucleotides does not have an adverse effect on RNAi activity. Replacing up to four nucleotides on each end of the siRNA with deoxyribonucleotides has been reported to be well tolerated, whereas complete substitution with deoxyribonucleotides results in no RNAi activity (Elbashir et al., 2001, EMBO J., 20, 6877 and Tuschl et al., International PCT Publication No. WO 01/75164). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 preliminarily suggest that siRNA may include modifications to either the phosphate-sugar backbone or the nucleoside to include at least one of a nitrogen or sulfur heteroatom, however, neither application postulates to what extent such modifications would be tolerated in siRNA molecules, nor provides any further guidance or examples of such modified siRNA. Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-O or 4′-C methylene bridge. However, Kreutzer at al. similarly fails to provide examples or guidance as to what extent these modifications would be tolerated in dsRNA molecules.

Parrish et al., 2000, Molecular Cell, 6, 1077-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081. The authors also tested certain modifications at the 2′-position of the nucleotide sugar in the long siRNA transcripts and found that substituting deoxynucleotides for ribonucleotides produced a substantial decrease in interference activity, especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl)uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 5-iodouracil and 3-(aminoallyl)uracil in the antisense strand resulted in a substantial decrease in RNAi activity as well.

The use of longer dsRNA has been described. For example, Beach et al., International PCT Publication No. WO 01/68836, describes specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due to the danger of activating interferon response. Li et al., International PCT Publication No. WO 00/44914, describe the use of specific long (141 bp-488 bp) enzymatically synthesized or vector expressed dsRNAs for attenuating the expression of certain target genes. Zernicka-Goetz et al., International PCT Publication No. WO 01/36646, describe certain methods for inhibiting the expression of particular genes in mammalian cells using certain long (550 bp-714 bp), enzymatically synthesized or vector expressed dsRNA molecules. Fire et al., International PCT Publication No. WO 99/32619, describe particular methods for introducing certain long dsRNA molecules into cells for use in inhibiting gene expression in nematodes. Plaetinck et al., International PCT Publication No. WO 00/01846, describe certain methods for identifying specific genes responsible for conferring a particular phenotype in a cell using specific long dsRNA molecules. Mello et al., International PCT Publication No. WO 01/29058, describe the identification of specific genes involved in dsRNA-mediated RNAi. Pachuck et al., International PCT Publication No. WO 00/63364, describe certain long (at least 200 nucleotide) dsRNA constructs. Deschamps Depaillette et al., International PCT Publication No. WO 99/07409, describe specific compositions consisting of particular dsRNA molecules combined with certain anti-viral agents. Waterhouse et al., International PCT Publication No. 99/53050 and 1998, PNAS, 95, 13959-13964, describe certain methods for decreasing the phenotypic expression of a nucleic acid in plant cells using certain dsRNAs. Driscoll et al., International PCT Publication No. WO 01/49844, describe specific DNA expression constructs for use in facilitating gene silencing in targeted organisms.

Others have reported on various RNAi and gene-silencing systems. For example, Parrish et al., 2000, Molecular Cell, 6, 1077-1087, describe specific chemically-modified dsRNA constructs targeting the unc-22 gene of C. elegans. Grossniklaus, International PCT Publication No. WO 01/38551, describes certain methods for regulating polycomb gene expression in plants using certain dsRNAs. Churikov et al., International PCT Publication No. WO 01/42443, describe certain methods for modifying genetic characteristics of an organism using certain dsRNAs. Cogoni et al., International PCT Publication No. WO 01/53475, describe certain methods for isolating a Neurospora silencing gene and uses thereof. Reed et al., International PCT Publication No. WO 01/68836, describe certain methods for gene silencing in plants. Honer et al., International PCT Publication No. WO 01/70944, describe certain methods of drug screening using transgenic nematodes as Parkinson's Disease models using certain dsRNAs. Deak et al., International PCT Publication No. WO 01/72774, describe certain Drosophila-derived gene products that may be related to RNAi in Drosophila. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313, describe certain methods and compositions for inhibiting the function of certain polynucleotide sequences using certain long (over 250 bp), vector expressed dsRNAs. Echeverri et al., International PCT Publication No. WO 02/38805, describe certain C. elegans genes identified via RNAi. Kreutzer et al., International PCT Publications Nos. WO 02/055692, WO 02/055693, and EP 1144623 B1 describes certain methods for inhibiting gene expression using dsRNA. Graham et al., International PCT Publications Nos. WO 99/49029 and WO 01/70949, and AU 4037501 describe certain vector expressed siRNA molecules. Fire et al., U.S. Pat. No. 6,506,559, describe certain methods for inhibiting gene expression in vitro using certain long dsRNA (299 bp-1033 bp) constructs that mediate RNAi. Martinez et al., 2002, Cell, 110, 563-574, describe certain single stranded siRNA constructs, including certain 5′-phosphorylated single stranded siRNAs that mediate RNA interference in Hela cells. Harborth et al., 2003, Antisense & Nucleic Acid Drug Development, 13, 83-105, describe certain chemically and structurally modified siRNA molecules. Chiu and Rana, 2003, RNA, 9, 1034-1048, describe certain chemically and structurally modified siRNA molecules. Woolf et al., International PCT Publication Nos. WO 03/064626 and WO 03/064625 describe certain chemically modified dsRNA constructs. Hornung et al., 2005, Nature Medicine, 11, 263-270, describe the sequence-specific potent induction of IFN-alpha by short interfering RNA in plasmacytoid dendritic cells through TLR7. Judge et al., 2005, Nature Biotechnology, Published online: 20 Mar. 2005, describe the sequence-dependent stimulation of the mammalian innate immune response by synthetic siRNA. Yuki et al., International PCT Publication Nos. WO 05/049821 and WO 04/048566, describe certain methods for designing short interfering RNA sequences and certain short interfering RNA sequences with optimized activity. Saigo et al., US Patent Application Publication No. US20040539332, describe certain methods of designing oligo- or polynucleotide sequences, including short interfering RNA sequences, for achieving RNA interference. Tei et al., International PCT Publication No. WO 03/044188, describe certain methods for inhibiting expression of a target gene, which comprises transfecting a cell, tissue, or individual organism with a double-stranded polynucleotide comprising DNA and RNA having a substantially identical nucleotide sequence with at least a partial nucleotide sequence of the target gene.

### BRIEF SUMMARY OF THE INVENTION

One aspect of the present invention provides an isolated small interfering RNA (siRNA) polynucleotide, comprising at least one nucleotide sequence selected from the group consisting of SEQ ID NOs:5-118. In one embodiment, the siRNA polynucleotide of the present invention comprises at least one nucleotide sequence selected from the group consisting of SEQ ID NOs:5-118 and the complementary polynucleotide thereto. In a further embodiment, the small interfering RNA polynucleotide inhibits expression of an ILK polypeptide, wherein the ILK polypeptide comprises an amino acid sequence as set forth in SEQ ID NOs:119-122, or that is encoded by the polynucleotide as set forth in any one of SEQ ID NOS:1-4. In another embodiment, the nucleotide sequence of the siRNA polynucleotide differs by one, two, three or four nucleotides at any positions of the siRNA polynucleotides as described herein, such as those provided in SEQ ID NOs: 5-118, or the complement thereof. In yet another embodiment, the nucleotide sequence of the siRNA polynucleotide differs by at least one mismatched base pair between a 5′ end of an antisense strand and a 3′ end of a sense strand of a sequence selected from the group consisting of the sequences set forth in SEQ ID NOS:5-118. In this regard, the mismatched base pair may include, but are not limited to G:A, C:A, C:U, G:G, A:A, C:C, U:U, C:T, and U:T mismatches. In a further embodiment, the mismatched base pair comprises a wobble base pair (e.g., G:U) between the 5′ end of the antisense strand and the 3′ end of the sense strand. In another embodiment, the siRNA polynucleotide comprises at least one synthetic nucleotide analogue of a naturally occurring nucleotide. In certain embodiments, wherein the siRNA polynucleotide is linked to a detectable label, such as a reporter molecule or a magnetic or paramagnetic particle. Reporter molecules are well known to the skilled artisan. Illustrative reporter molecules include, but are in no way limited to, a dye, a radionuclide, a luminescent group, a fluorescent group, and biotin.

Another aspect of the invention provides an isolated siRNA molecule that inhibits expression of an ILK gene, wherein the siRNA molecule comprises a nucleic acid that targets the sequence provided in SEQ ID NOs:1-4, or a variant thereof having kinase activity, in particular the ability to bind to and phosphorylate the β1-integrin cytoplasmic domain. In certain embodiments, the siRNA comprises any one of the single stranded RNA sequences provided in SEQ ID NOs:5-118, or a double-stranded RNA thereof. In one embodiment of the invention, the siRNA molecule down regulates expression of an ILK gene via RNA interference (RNAi).

Another aspect of the invention provides compositions comprising any one or more of the siRNA polynucleotides described herein and a physiologically acceptable carrier. In certain embodiments, the composition comprises polyethyleneimine. In another embodiment, the composition comprises polyethyleneimine and NHS-PEG-VS. In a further embodiment, the composition comprises a positively charged polypeptide. In this regard, the positively charged polypeptide may comprise a poly(Histidine-Lysine). In a further embodiment, the composition further comprises a targeting moiety.

Another aspect of the invention provides a method for treating or preventing a number of diseases such as those described herein in a subject having or suspected of being at risk for having such a disease, comprising administering to the subject a composition of the invention, such as a composition comprising the siRNa molecules of the invention, thereby treating or preventing the disease. The present invention provides application of the siRNA molecules of the present invention as therapeutic agents for human diseases such as cancers including, brain, esophageal, bladder, cervical, breast, lung, prostate, stomach, colorectal, pancreatic, head and neck, prostate, thyroid, kidney, or ovarian cancer, or melanoma, lymphoma, glioma, or glioblastoma; metabolic disorders and inflammatory diseases, such as but not limited to asthma, Chronic Obstructive Pulmonary Disease (COPD), inflammatory bowel disease, ankylosing spondylitis, Reiter's syndrome, Crohn's disease, ulcerative colitis, systemic lupus erythematosus, psoriasis, artherosclerosis, rheumatoid arthritis, osteoarthritis, or multiple sclerosis; diabetes mellitus, hyperlipidemia, lactic acidosis, phenylketonuria, tyrosinemias, alcaptonurta, isovaleric acidemia, homocystinuria, urea cycle disorders, or an organic acid metabolic disorder, propionic acidemia, methylmalonic acidemia, glutaric aciduria Type 1, acid lipase disease, amyloidosis, Barth syndrome, biotinidase deficiency (BD), carnitine palitoyl transferase deficiency type II (CPT-II), central pontine myelinolysis, muscular dystrophy, Farber's disease, G6PD deficiency (Glucose-6-Phosphate Dehydrogenase), gangliosidoses, trimethylaminuria, Lesch-Nyhan syndrome, lipid storage diseases, metabolic myopathies, methylmalonic aciduria (MMA), mitochondrial myopathies, MPS (Mucopolysaccharidoses) and related diseases, mucolipidoses, mucopolysaccharidoses, multiple CoA carboxylase deficiency (MCCD), nonketotic hyperglycinemia, Pompe disease, propionic acidemia (PROP), and Type I glycogen storage disease; diseases associated with abnormal or pathological angiogenesis, such as, but not limited to, psoriasis and age-related macular degeneration.

A further aspect of the invention provides a method for inhibiting the synthesis or expression of ILK comprising contacting a cell expressing ILK with any one or more siRNA molecules wherein the one or more siRNA molecules comprises a sequence selected from the sequences provided in SEQ ID NOs:5-118, or a double-stranded RNA thereof. In one embodiment, a nucleic acid sequence encoding ILK comprises the sequence set forth in any one of SEQ ID NOS:1-4.

Yet a further aspect of the invention provides a method for reducing the severity of any of the disease described herein as related to ILK expression in a subject having the disease, comprising administering to the subject a composition comprising the siRNA as described herein, thereby reducing the severity of the disease.

Another aspect of the invention provides a recombinant nucleic acid construct comprising a nucleic acid that is capable of directing transcription of a small interfering RNA (siRNA), the nucleic acid comprising: (a) a first promoter; (b) a second promoter; and (c) at least one DNA polynucleotide segment comprising at least one polynucleotide that is selected from the group consisting of (i) a polynucleotide comprising the nucleotide sequence set forth in any one of SEQ ID NOs:5-118, and (ii) a polynucleotide of at least 18 nucleotides that is complementary to the polynucleotide of (i), wherein the DNA polynucleotide segment is operably linked to at least one of the first and second promoters, and wherein the promoters are oriented to direct transcription of the DNA polynucleotide segment and of the complement thereto. In one embodiment, the recombinant nucleic acid construct comprises at least one enhancer that is selected from a first enhancer operably linked to the first promoter and a second enhancer operably linked to the second promoter. In another embodiment, the recombinant nucleic acid construct comprises at least one transcriptional terminator that is selected from (i) a first transcriptional terminator that is positioned in the construct to terminate transcription directed by the first promoter and (ii) a second transcriptional terminator that is positioned in the construct to terminate transcription directed by the second promoter.

Another aspect of the invention provides isolated host cells transformed or transfected with a recombinant nucleic acid construct as described herein.

One aspect of the present invention provides a nucleic acid molecule that down regulates expression of ILK, wherein the nucleic acid molecule comprises a nucleic acid that targets ILK mRNA, whose representative sequences are provided in SEQ ID NOs:1-4. Corresponding amino acid sequences are set forth in SEQ ID NOs:119-122. In one embodiment, the nucleic acid is an siRNA molecule. In a further embodiment, the siRNA comprises any one of the single stranded RNA sequences provided in SEQ ID NOs:5-118, or a double-stranded RNA thereof. In another embodiment, the nucleic acid molecule down regulates expression of ILK gene via RNA interference (RNAi).

A further aspect of the invention provides a composition comprising any one or more of the siRNA molecules of the invention as set forth in SEQ ID NOs:5-118. In this regard, the composition may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more siRNA molecules of the invention. In certain embodiments, the siRNA molecules may all target the ILK gene, or a combination of ILK and one or more other targets, such as polynucleotides that encode proteins involved in the LEF-1/beta catenin signaling pathway, the Wnt signaling cascade (Novak et al., Proc. Natl. Acad. Sci. U.S.A., 1998, 95, 4374-4379), G-proteins (Tu et al., Mol. Cell. Biol., 1999, 19, 2425-2434) phosphotidylinositol 3-kinase, protein kinase B and glycogen synthase kinase 3 (Delcommenne et al., Proc. Natl. Acad. Sci. U.S.A., 1998, 95, 11211-11216). In this regard, the siRNA molecules may be selected from the siRNA molecules provided in SEQ ID NOs:5-118, or a double-stranded RNA thereof. Thus, the siRNA molecules may target ILK and may be a mixture of siRNA molecules that target different regions of this gene. In certain embodiments, the compositions may comprise a targeting moiety or ligand, such as a targeting moeity that will target the siRNA composition to a desired cell.

These and other aspects of the present invention will become apparent upon reference to the following detailed description.

### DETAILED DESCRIPTION OF THE INVENTION

The present invention relates to nucleic acid molecules for modulating the expression of ILK. In certain embodiments the nucleic acid is ribonucleic acid (RNA). In certain embodiments, the RNA molecules are single or double stranded. In this regard, the nucleic acid based molecules of the present invention, such as siRNA, inhibit or down-regulate expression of ILK.

The present invention relates to compounds, compositions, and methods for the study, diagnosis, and treatment of traits, diseases and conditions that respond to the modulation of ILK gene expression and/or activity. The present invention is also directed to compounds, compositions, and methods relating to traits, diseases and conditions that respond to the modulation of expression and/or activity of genes involved in ILK gene expression pathways or other cellular processes that mediate the maintenance or development of such traits, diseases and conditions. Specifically, the invention relates to double stranded nucleic acid molecules including small nucleic acid molecules, such as short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against ILK gene expression, including cocktails of such small nucleic acid molecules and nanoparticle formulations of such small nucleic acid molecules. The present invention also relates to small nucleic acid molecules, such as siNA, siRNA, and others that can inhibit the function of endogenous RNA molecules, such as endogenous micro-RNA (miRNA) (e.g, miRNA inhibitors) or endogenous short interfering RNA (siRNA), (e.g., siRNA inhibitors) or that can inhibit the function of RISC (e.g., RISC inhibitors), to modulate ILK gene expression by interfering with the regulatory function of such endogenous RNAs or proteins associated with such endogenous RNAs (e.g., RISC), including cocktails of such small nucleic acid molecules and nanoparticle formulations of such small nucleic acid molecules. Such small nucleic acid molecules are useful, for example, in providing compositions to prevent, inhibit, or reduce cancers including, brain, esophageal, bladder, cervical, breast, lung, prostate, stomach, colorectal, pancreatic, head and neck, prostate, thyroid, kidney, or ovarian cancer, or melanoma, lymphoma, glioma, or glioblastoma; metabolic disorders and inflammatory diseases, such as but not limited to asthma, Chronic Obstructive Pulmonary Disease (COPD), inflammatory bowel disease, ankylosing spondylitis, Reiter's syndrome, Crohn's disease, ulcerative colitis, systemic lupus erythematosus, psoriasis, artherosclerosis, rheumatoid arthritis, osteoarthritis, or multiple sclerosis; diabetes mellitus, hyperlipidemia, lactic acidosis, phenylketonuria, tyrosinemias, alcaptonurta, isovaleric acidemia, homocystinuria, urea cycle disorders, or an organic acid metabolic disorder, propionic acidemia, methylmalonic acidemia, glutaric aciduria Type 1, acid lipase disease, amyloidosis, Barth syndrome, biotinidase deficiency (BD), carnitine palitoyl transferase deficiency type II (CPT-II), central pontine myelinolysis, muscular dystrophy, Farber's disease, G6PD deficiency (Glucose-6-Phosphate Dehydrogenase), gangliosidoses, trimethylaminuria, Lesch-Nyhan syndrome, lipid storage diseases, metabolic myopathies, methylmalonic aciduria (MMA), mitochondrial myopathies, MPS (Mucopolysaccharidoses) and related diseases, mucolipidoses, mucopolysaccharidoses, multiple CoA carboxylase deficiency (MCCD), nonketotic hyperglycinemia, Pompe disease, propionic acidemia (PROP), and Type I glycogen storage disease; diseases associated with abnormal or pathological angiogenesis, such as, but not limited to, psoriasis and age-related macular degeneration; and/or other disease states, conditions, or traits associated with ILK gene expression or activity in a subject or organism.

By “inhibit” or “down-regulate” it is meant that the expression of the gene, or level of mRNA encoding an ILK protein, levels of ILK protein, or activity of ILK, is reduced below that observed in the absence of the nucleic acid molecules of the invention. In one embodiment, inhibition or down-regulation with the nucleic acid molecules of the invention is below that level observed in the presence of an inactive control or attenuated molecule that is able to bind to the same target mRNA, but is unable to cleave or otherwise silence that mRNA. In another embodiment, inhibition or down-regulation with the nucleic acid molecules of the invention is preferably below that level observed in the presence of, for example, a nucleic acid with scrambled sequence or with appropriately disruptive mismatches. In another embodiment, inhibition or down-regulation of ILK with the nucleic acid molecule of the instant invention is greater in the presence of the nucleic acid molecule than in its absence.

By “modulate” is meant that the expression of the gene, or level of RNAs or equivalent RNAs encoding one or more protein subunits, or activity of one or more protein subunit(s) is up-regulated or down-regulated, such that the expression, level, or activity is greater than or less than that observed in the absence of the nucleic acid molecules of the invention.

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as small interfering RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein also refers to a double stranded RNA molecule capable of mediating RNA interference “RNAi”, including small interfering RNA “siRNA” (see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914).

By “gene” it is meant a nucleic acid that encodes an RNA, for example, nucleic acid sequences including but not limited to structural genes encoding a polypeptide.

By “a nucleic acid that targets” is meant a nucleic acid as described herein that matches, is complementary to or otherwise specifically binds or specifically hybridizes to and thereby can modulate the expression of the gene that comprises the target sequence, or level of mRNAs or equivalent RNAs encoding one or more protein subunits, or activity of one or more protein subunit(s) encoded by the gene.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII, pp. 123-133; Frier et al., 1986, Proc. Nat. Acad. Sci. USA 83, 9373-9377; Turner et al., 1987, J. Am. Chem. Soc. 109, 3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “RNA interference” or “RNAi” is meant a biological process of inhibiting or down regulating gene expression in a cell as is generally known in the art and which is mediated by short interfering nucleic acid molecules, see for example Zamore and Haley, 2005, Science, 309, 1519-1524; Vaughn and Martienssen, 2005, Science, 309, 1525-1526; Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zemicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). In addition, as used herein, the term RNAi is meant to be equivalent to other terms used to describe sequence specific RNA interference, such as post transcriptional gene silencing, translational inhibition, transcriptional inhibition, or epigenetics. For example, siRNA molecules of the invention can be used to epigenetically silence genes at both the post-transcriptional level or the pre-transcriptional level. In a non-limiting example, epigenetic modulation of gene expression by siRNA molecules of the invention can result from siRNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siRNA molecules of the invention can result from siRNA mediated cleavage of RNA (either coding or non-coding RNA) via RISC, or alternately, translational inhibition as is known in the art. In another embodiment, modulation of gene expression by siRNA molecules of the invention can result from transcriptional inhibition (see for example Janowski et al., 2005, Nature Chemical Biology, 1, 216-222).

Two types of about 21 nucleotide RNAs trigger post-transcriptional gene silencing in animals: small interfering RNAs (siRNAs) and microRNAs (miRNAs). Both siRNAs and miRNAs are produced by the cleavage of double-stranded RNA (dsRNA) precursors by Dicer, a nuclease of the RNase III family of dsRNA-specific endonucleases (Bernstein et al., (2001). Nature 409, 363-366; Billy, E., et al. (2001). Proc Natl Acad Sci USA 98, 14428-14433; Grishok et al., 2001, Cell 106, 23-34; Hutvgner et al., 2001, Science 293, 834-838; Ketting et al., 2001, Genes Dev 15, 2654-2659; Knight and Bass, 2001, Science 293, 2269-2271; Paddison et al., 2002, Genes Dev 16, 948-958; Park et al., 2002, Curr Biol 12, 1484-1495; Provost et al., 2002, EMBO J. 21, 5864-5874; Reinhart et al., 2002, Science. 297: 1831; Zhang et al., 2002, EMBO J. 21, 5875-5885; Doi et al., 2003, Curr Biol 13, 41-46; Myers et al., 2003, Nature Biotechnology Mar; 21(3):324-8). siRNAs result when transposons, viruses or endogenous genes express long dsRNA or when dsRNA is introduced experimentally into plant or animal cells to trigger gene silencing, also called RNA interference (RNAi) (Fire et al., 1998; Hamilton and Baulcombe, 1999; Zamore et al., 2000; Elbashir et al., 2001 a; Hammond et al., 2001; Sijen et al., 2001; Catalanotto et al., 2002). In contrast, miRNAs are the products of endogenous, non-coding genes whose precursor RNA transcripts can form small stem-loops from which mature miRNAs are cleaved by Dicer (Lagos-Quintana et al., 2001; Lau et al., 2001; Lee and Ambros, 2001; Lagos-Quintana et al., 2002; Mourelatos et al., 2002; Reinhart et al., 2002; Ambros et al., 2003; Brennecke et al., 2003; Lagos-Quintana et al., 2003; Lim et al., 2003a; Lim et al., 2003b). miRNAs are encoded by genes distinct from the mRNAs whose expression they control.

siRNAs were first identified as the specificity determinants of the RNA interference (RNAi) pathway (Hamilton and Baulcombe, 1999; Hammond et al., 2000), where they act as guides to direct endonucleolytic cleavage of their target RNAs (Zamore et al., 2000; Elbashir et al., 2001a). Prototypical siRNA duplexes are 21 nt, double-stranded RNAs that contain 19 base pairs, with two-nucleotide, 3′ overhanging ends (Elbashir et al., 2001a; Nyknen et al., 2001; Tang et al., 2003). Active siRNAs contain 5′ phosphates and 3′ hydroxyls (Zamore et al., 2000; Boutla et al., 2001; Nyknen et al., 2001; Chiu and Rana, 2002). Similarly, miRNAs contain 5′ phosphate and 3′ hydroxyl groups, reflecting their production by Dicer (Hutvgner et al., 2001; Mallory et al., 2002)

Thus, the present invention is directed in part to the discovery of short RNA polynucleotide sequences that are capable of specifically modulating expression of a target ILK polypeptide, such as encoded by the sequence provided in SEQ ID NOs:1-4, or a variant thereof. Illustrative siRNA polynucleotide sequences that specifically modulate the expression of ILK are provided in SEQ ID NOs:5-118. Without wishing to be bound by theory, the RNA polynucleotides of the present invention specifically reduce expression of a desired target polypeptide through recruitment of small interfering RNA (siRNA) mechanisms. In particular, and as described in greater detail herein, according to the present invention there are provided compositions and methods that relate to the identification of certain specific RNAi oligonucleotide sequences of 19, 20, 21, 22, 23, 24, 25, 26 or 27 nucleotides that can be derived from corresponding polynucleotide sequences encoding the desired ILK target polypeptide.

In certain embodiments of the invention, the siRNA polynucleotides interfere with expression of a ILK target polypeptide or a variant thereof, and comprises a RNA oligonucleotide or RNA polynucleotide uniquely corresponding in its nucleotide base sequence to the sequence of a portion of a target polynucleotide encoding the target polypeptide, for instance, a target mRNA sequence or an exonic sequence encoding such mRNA. The invention relates in certain embodiments to siRNA polynucleotides that interfere with expression (sometimes referred to as silencing) of specific polypeptides in mammals, which in certain embodiments are humans and in certain other embodiments are non-human mammals. Hence, according to non-limiting theory, the siRNA polynucleotides of the present invention direct sequence-specific degradation of mRNA encoding a desired target polypeptide, such as ILK.

In certain embodiments, the term “siRNA” means either: (i) a double stranded RNA oligonucleotide, or polynucleotide, that is 18 base pairs, 19 base pairs, 20 base pairs, 21 base pairs, 22 base pairs, 23 base pairs, 24 base pairs, 25 base pairs, 26 base pairs, 27 base pairs, 28 base pairs, 29 base pairs or 30 base pairs in length and that is capable of interfering with expression and activity of a ILK polypeptide, or a variant of the ILK polypeptide, wherein a single strand of the siRNA comprises a portion of a RNA polynucleotide sequence that encodes the ILK polypeptide, its variant, or a complementary sequence thereto; (ii) a single stranded oligonucleotide, or polynucleotide of 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides or 30 nucleotides in length and that is either capable of interfering with expression and/or activity of a target ILK polypeptide, or a variant of the ILK polypeptide, or that anneals to a complementary sequence to result in a dsRNA that is capable of interfering with target polypeptide expression, wherein such single stranded oligonucleotide comprises a portion of a RNA polynucleotide sequence that encodes the ILK polypeptide, its variant, or a complementary sequence thereto; or (iii) an oligonucleotide, or polynucleotide, of either (i) or (ii) above wherein such oligonucleotide, or polynucleotide, has one, two, three or four nucleic acid alterations or substitutions therein. Certain RNAi oligonucleotide sequences described herein are complementary to the 3′ non-coding region of target mRNA that encodes the ILK polypeptide.

A siRNA polynucleotide is a RNA nucleic acid molecule that mediates the effect of RNA interference, a post-transcriptional gene silencing mechanism. In certain embodiments, a siRNA polynucleotide comprises a double-stranded RNA (dsRNA) but is not intended to be so limited and may comprise a single-stranded RNA (see, e.g., Martinez et al. Cell 110:563-74 (2002)). A siRNA polynucleotide may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the subject invention siRNA polynucleotides are also intended to describe the corresponding RNA sequences and their complements, given the well established principles of complementary nucleotide base-pairing. A siRNA may be transcribed using as a template a DNA (genomic, cDNA, or synthetic) that contains a RNA polymerase promoter, for example, a U6 promoter or the H1 RNA polymerase III promoter, or the siRNA may be a synthetically derived RNA molecule. In certain embodiments the subject invention siRNA polynucleotide may have blunt ends, that is, each nucleotide in one strand of the duplex is perfectly complementary (e.g., by Watson-Crick base-pairing) with a nucleotide of the opposite strand. In certain other embodiments, at least one strand of the subject invention siRNA polynucleotide has at least one, and in certain embodiments, two nucleotides that “overhang” (i.e., that do not base pair with a complementary base in the opposing strand) at the 3′ end of either strand, or in certain embodiments, both strands, of the siRNA polynucleotide. In one embodiment of the invention, each strand of the siRNA polynucleotide duplex has a two-nucleotide overhang at the 3′ end. The two-nucleotide overhang may be a thymidine dinucleotide (TT) but may also comprise other bases, for example, a TC dinucleotide or a TG dinucleotide, or any other dinucleotide. For a discussion of 3′ ends of siRNA polynucleotides see, e.g., WO 01/75164.

Certain illustrative siRNA polynucleotides comprise double-stranded oligomeric nucleotides of about 18-30 nucleotide base pairs. In certain embodiments, the siRNA molecules of the invention comprise about 18, 19, 20, 21, 22, 23, 24, 25, 26, or 27 base pairs, and in other particular embodiments about 19, 20, 21, 22 or 23 base pairs, or about 27 base pairs, whereby the use of “about” indicates, as described above, that in certain embodiments and under certain conditions the processive cleavage steps that may give rise to functional siRNA polynucleotides that are capable of interfering with expression of a selected polypeptide may not be absolutely efficient. Hence, siRNA polynucleotides, for instance, of “about” 18, 19, 20, 21, 22, 23, 24, or 25 base pairs may include one or more siRNA polynucleotide molecules that may differ (e.g., by nucleotide insertion or deletion) in length by one, two, three or four base pairs, by way of non-limiting theory as a consequence of variability in processing, in biosynthesis, or in artificial synthesis. The contemplated siRNA polynucleotides of the present invention may also comprise a polynucleotide sequence that exhibits variability by differing (e.g., by nucleotide substitution, including transition or transversion) at one, two, three or four nucleotides from a particular sequence, the differences occurring at any of positions 1, 2, 3, 4, 5, 6, 7, 8, 9, 10, 11, 12, 13, 14, 15, 16, 17, 18, or 19 of a particular siRNA polynucleotide sequence, or at positions 20, 21, 22, 23, 24, 25, 26, or 27 of siRNA polynucleotides depending on the length of the molecule, whether situated in a sense or in an antisense strand of the double-stranded polynucleotide. The nucleotide substitution may be found only in one strand, by way of example in the antisense strand, of a double-stranded polynucleotide, and the complementary nucleotide with which the substitute nucleotide would typically form hydrogen bond base pairing may not necessarily be correspondingly substituted in the sense strand. In certain embodiments, the siRNA polynucleotides are homogeneous with respect to a specific nucleotide sequence. As described herein, the siRNA polynucleotides interfere with expression of an ILK polypeptide. These polynucleotides may also find uses as probes or primers.

In certain embodiments, the efficacy and specificity of gene/protein silencing by the siRNA nucleic acids of the present invention may be enhanced using the methods described in US Patent Application Publications 2005/0186586, 2005/0181382, 2005/0037988, and 2006/0134787. In this regard, the RNA silencing may be enhanced by lessening the base pair strength between the 5′ end of the first strand and the 3′ end of a second strand of the duplex as compared to the base pair strength between the 3′ end of the first strand and the 5′ end of the second strand. In certain embodiments the RNA duplex may comprise at least one blunt end and may comprise two blunt ends. In other embodiments, the duplex comprises at least one overhang and may comprise two overhangs.

In one embodiment of the invention, the ability of the siRNA molecule to silence a target gene is enhanced by enhancing the ability of a first strand of a RNAi agent to act as a guide strand in mediating RNAi. This is achieved by lessening the base pair strength between the 5′ end of the first strand and the 3′ end of a second strand of the duplex as compared to the base pair strength between the 3′ end of the first strand and the 5′ end of the second strand.

In a further aspect of the invention, the efficacy of a siRNA duplex is enhanced by lessening the base pair strength between the antisense strand 5′ end (AS 5′) and the sense strand 3′ end (S 3′) as compared to the base pair strength between the antisense strand 3′ end (AS 3′) and the sense strand 5′ end (S ′5), such that efficacy is enhanced.

In certain embodiments, modifications can be made to the siRNA molecules of the invention in order to promote entry of a desired strand of an siRNA duplex into a RISC complex. This is achieved by enhancing the asymmetry of the siRNA duplex, such that entry of the desired strand is promoted. In this regard, the asymmetry is enhanced by lessening the base pair strength between the 5′ end of the desired strand and the 3′ end of a complementary strand of the duplex as compared to the base pair strength between the 3′ end of the desired strand and the 5′ end of the complementary strand. In certain embodiments, the base-pair strength is less due to fewer G:C base pairs between the 5′ end of the first or antisense strand and the 3′ end of the second or sense strand than between the 3′ end of the first or antisense strand and the 5′ end of the second or sense strand. In other embodiments, the base pair strength is less due to at least one mismatched base pair between the 5′ end of the first or antisense strand and the 3′ end of the second or sense strand. In certain embodiments, the mismatched base pairs include but are not limited to G:A, C:A, C:U, G:G, A:A, C:C, U:U, C:T, and U:T. In one embodiment, the base pair strength is less due to at least one wobble base pair between the 5′ end of the first or antisense strand and the 3′ end of the second or sense strand. In this regard, the wobble base pair may be G:U. or G:T.

In certain embodiments, the base pair strength is less due to: (a) at least one mismatched base pair between the 5′ end of the first or antisense strand and the 3′ end of the second or sense strand; and (b) at least one wobble base pair between the 5′ end of the first or antisense strand and the 3′ end of the second or sense strand. Thus, the mismatched base pair may be selected from the group consisting of G:A, C:A, C:U, G:G, A:A, C:C and U:U. In another embodiment, the mismatched base pair is selected from the group consisting of G:A, C:A, C:T, G:G, A:A, C:C and U:T. In certain cases, the wobble base pair is G:U or G:T.

In certain embodiments, the base pair strength is less due to at least one base pair comprising a rare nucleotide such as inosine, 1-methyl inosine, pseudouridine, 5,6-dihydrouridine, ribothymidine, 2N-methylguanosine and 2,2N,N-dimethylguanosine; or a modified nucleotide, such as 2-amino-G, 2-amino-A, 2,6-diamino-G, and 2,6-diamino-A.

As used herein, the term “antisense strand” of an siRNA or RNAi agent refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific RNA interference (RNAi), e.g., complementarity sufficient to trigger the destruction of the desired target mRNA by the RNAi machinery or process. The term “sense strand” or “second strand” of an siRNA or RNAi agent refers to a strand that is complementary to the antisense strand or first strand. Antisense and sense strands can also be referred to as first or second strands, the first or second strand having complementarity to the target sequence and the respective second or first strand having complementarity to said first or second strand.

As used herein, the term “guide strand” refers to a strand of an RNAi agent, e.g., an antisense strand of an siRNA duplex, that enters into the RISC complex and directs cleavage of the target mRNA.

Thus, complete complementarity of the siRNA molecules of the invention with their target gene is not necessary in order for effective silencing to occur. In particular, three or four mismatches between a guide strand of an siRNA duplex and its target RNA, properly placed so as to still permit mRNA cleavage, facilitates the release of cleaved target RNA from the RISC complex, thereby increasing the rate of enzyme turnover. In particular, the efficiency of cleavage is greater when a G:U base pair, referred to also as a G:U wobble, is present near the 5′ or 3′ end of the complex formed between the miRNA and the target.

Thus, at least one terminal nucleotide of the RNA molecules described herein can be substituted with a nucleotide that does not form a Watson-Crick base pair with the corresponding nucleotide in a target mRNA.

Polynucleotides that are siRNA polynucleotides of the present invention may in certain embodiments be derived from a single-stranded polynucleotide that comprises a single-stranded oligonucleotide fragment (e.g., of about 18-30 nucleotides, which should be understood to include any whole integer of nucleotides including and between 18 and 30) and its reverse complement, typically separated by a spacer sequence. According to certain such embodiments, cleavage of the spacer provides the single-stranded oligonucleotide fragment and its reverse complement, such that they may anneal to form (optionally with additional processing steps that may result in addition or removal of one, two, three or more nucleotides from the 3′ end and/or the 5′ end of either or both strands) the double-stranded siRNA polynucleotide of the present invention. In certain embodiments the spacer is of a length that permits the fragment and its reverse complement to anneal and form a double-stranded structure (e.g., like a hairpin polynucleotide) prior to cleavage of the spacer (and, optionally, subsequent processing steps that may result in addition or removal of one, two, three, four, or more nucleotides from the 3′ end and/or the 5′ end of either or both strands). A spacer sequence may therefore be any polynucleotide sequence as provided herein that is situated between two complementary polynucleotide sequence regions which, when annealed into a double-stranded nucleic acid, comprise a siRNA polynucleotide. In some embodiments, a spacer sequence comprises at least 4 nucleotides, although in certain embodiments the spacer may comprise 5, 6, 7, 8, 9, 10, 11, 12, 13, 14, 15, 16,17, 18, 19, 20, 21-25, 26-30, 31-40, 41-50, 51-70, 71-90, 91-110, 111-150, 151-200 or more nucleotides. Examples of siRNA polynucleotides derived from a single nucleotide strand comprising two complementary nucleotide sequences separated by a spacer have been described (e.g., Brummelkamp et al., 2002 Science 296:550; Paddison et al., 2002 Genes Develop. 16:948; Paul et al. Nat. Biotechnol. 20:505-508 (2002); Grabarek et al., BioTechniques 34:734-44 (2003)).

Polynucleotide variants may contain one or more substitutions, additions, deletions, and/or insertions such that the activity of the siRNA polynucleotide is not substantially diminished, as described above. The effect on the activity of the siRNA polynucleotide may generally be assessed as described herein or using conventional methods. In certain embodiments, variants exhibit at least about 75%, 78%, 80%, 85%, 87%, 88% or 89% identity and in particular embodiments, at least about 90%, 92%, 95%, 96%, 97%, 98%, or 99% identity to a portion of a polynucleotide sequence that encodes a native ILK. The percent identity may be readily determined by comparing sequences of the polynucleotides to the corresponding portion of a full-length ILK polynucleotide such as those known to the art and cited herein, using any method including using computer algorithms well known to those having ordinary skill in the art, such as Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (see [online] Internet:<URL: ncbi dot nlm dot nih dot gov/cgi-bin/BLAST). Default parameters may be used.

Certain siRNA polynucleotide variants are substantially homologous to a portion of a native ILK gene. Single-stranded nucleic acids derived (e.g., by thermal denaturation) from such polynucleotide variants are capable of hybridizing under moderately stringent conditions or stringent conditions to a naturally occurring DNA or RNA sequence encoding a native ILK polypeptide (or a complementary sequence). A polynucleotide that detectably hybridizes under moderately stringent conditions or stringent conditions may have a nucleotide sequence that includes at least 10 consecutive nucleotides, 11, 12, 13, 14, 15, 16, 17, 18, 19, 20, 21, 22, 23, 24, 25, 26, 27, 28, 29 or 30 consecutive nucleotides complementary to a particular polynucleotide. In certain embodiments, such a sequence (or its complement) will be unique to an ILK polypeptide for which interference with expression is desired, and in certain other embodiments the sequence (or its complement) may be shared by ILK and one or more related polypeptides for which interference with polypeptide expression is desired.

Suitable moderately stringent conditions and stringent conditions are known to the skilled artisan. Moderately stringent conditions include, for example, pre-washing in a solution of 5×SSC, 0.5% SDS, 1.0 mM EDTA (pH 8.0); hybridizing at 50° C.-70° C., 5×SSC for 1-16 hours (e.g., overnight); followed by washing once or twice at 22-65° C. for 20-40 minutes with one or more each of 2×, 0.5× and 0.2×SSC containing 0.05-0.1% SDS. For additional stringency, conditions may include a wash in 0.1×SSC and 0.1% SDS at 50-60° C. for 15-40 minutes. As known to those having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used, and of the blotted, proband nucleic acid sample. Accordingly, it will be appreciated that suitably stringent conditions can be readily selected without undue experimentation when a desired selectivity of the probe is identified, based on its ability to hybridize to one or more certain proband sequences while not hybridizing to certain other proband sequences.

Sequence specific siRNA polynucleotides of the present invention may be designed using one or more of several criteria. For example, to design a siRNA polynucleotide that has 19 consecutive nucleotides identical to a sequence encoding a polypeptide of interest (e.g., ILK and other polypeptides described herein), the open reading frame of the polynucleotide sequence may be scanned for 21-base sequences that have one or more of the following characteristics: (1) an A+T/G+C ratio of approximately 1:1 but no greater than 2:1 or 1:2; (2) an AA dinucleotide or a CA dinucleotide at the 5′ end; (3) an internal hairpin loop melting temperature less than 55° C.; (4) a homodimer melting temperature of less than 37° C. (melting temperature calculations as described in (3) and (4) can be determined using computer software known to those skilled in the art); (5) a sequence of at least 16 consecutive nucleotides not identified as being present in any other known polynucleotide sequence (such an evaluation can be readily determined using computer programs available to a skilled artisan such as BLAST to search publicly available databases). Alternatively, an siRNA polynculeotide sequence may be designed and chosen using a computer software available commercially from various vendors (e.g., OligoEngine™ (Seattle, Wash.); Dharmacon, Inc. (Lafayette, Colo.); Ambion Inc. (Austin, Tex.); and QIAGEN, Inc. (Valencia, Calif.)). (See also Elbashir et al., Genes & Development 15:188-200 (2000); Elbashir et al., Nature 411:494-98 (2001)) The siRNA polynucleotides may then be tested for their ability to interfere with the expression of the target polypeptide according to methods known in the art and described herein. The determination of the effectiveness of an siRNA polynucleotide includes not only consideration of its ability to interfere with polypeptide expression but also includes consideration of whether the siRNA polynucleotide manifests undesirably toxic effects, for example, apoptosis of a cell for which cell death is not a desired effect of RNA interference (e.g., interference of ILK expression in a cell).

In certain embodiments, the nucleic acid inhibitors comprise sequences which are complementary to any known ILK sequence, including variants thereof that have altered expression and/or activity, particularly variants associated with disease. Variants of ILK include sequences having 70%, 75%, 80%, 85%, 90%, 91%, 92%, 93%, 94%, 95%, 96%, 97%, 98%, 99% or higher sequence identity to the wild type ILK sequences, such as those set forth in SEQ ID NOs:1-4 where such variants of ILK may demonstrate altered (increased or decreased) kinase activity, in particular the ability to bind to and phosphorylate the β1-integrin cytoplasmic domain. As would be understood by the skilled artisan, ILK sequences are available in any of a variety of public sequence databases including GENBANK or SWISSPROT. In one embodiment, the nucleic acid inhibitors (e.g., siRNA) of the invention comprise sequences complimentary to the specific ILK target sequences provided in SEQ ID NOs:1-4, or polynucleotides encoding the amino acid sequences provided in SEQ ID NOs:119-122. Examples of such siRNA molecules also are shown in the Examples and provided in SEQ ID NOs:5-118.

Polynucleotides, including target polynucleotides (e.g., polynucleotides capable of encoding a target polypeptide of interest), may be prepared using any of a variety of techniques, which will be useful for the preparation of specifically desired siRNA polynucleotides and for the identification and selection of desirable sequences to be used in siRNA polynucleotides. For example, a polynucleotide may be amplified from cDNA prepared from a suitable cell or tissue type. Such polynucleotides may be amplified via polymerase chain reaction (PCR). For this approach, sequence-specific primers may be designed based on the sequences provided herein and may be purchased or synthesized. An amplified portion may be used to isolate a full-length gene, or a desired portion thereof, from a suitable library using well known techniques. Within such techniques, a library (cDNA or genomic) is screened using one or more polynucleotide probes or primers suitable for amplification. In certain embodiments, a library is size-selected to include larger molecules. Random primed libraries may also be preferred for identifying 5′ and upstream regions of genes. Genomic libraries are preferred for obtaining introns and extending 5′ sequences. Suitable sequences for a siRNA polynucleotide contemplated by the present invention may also be selected from a library of siRNA polynucleotide sequences.

For hybridization techniques, a partial sequence may be labeled (e.g., by nick-translation or end-labeling with ³²P) using well known techniques. A bacterial or bacteriophage library may then be screened by hybridizing filters containing denatured bacterial colonies (or lawns containing phage plaques) with the labeled probe (see, e.g., Sambrook et al., Molecular Cloning: A Laboratory Manual, Cold Spring Harbor Laboratories, Cold Spring Harbor, N.Y., 2001). Hybridizing colonies or plaques are selected and expanded, and the DNA is isolated for further analysis. Clones may be analyzed to determine the amount of additional sequence by, for example, PCR using a primer from the partial sequence and a primer from the vector. Restriction maps and partial sequences may be generated to identify one or more overlapping clones. A full-length cDNA molecule can be generated by ligating suitable fragments, using well known techniques.

Alternatively, numerous amplification techniques are known in the art for obtaining a full-length coding sequence from a partial cDNA sequence. Within such techniques, amplification is generally performed via PCR. One such technique is known as “rapid amplification of cDNA ends” or RACE. This technique involves the use of an internal primer and an external primer, which hybridizes to a polyA region or vector sequence, to identify sequences that are 5′ and 3′ of a known sequence. Any of a variety of commercially available kits may be used to perform the amplification step. Primers may be designed using, for example, software well known in the art. Primers (or oligonucleotides for other uses contemplated herein, including, for example, probes and antisense oligonucleotides) are generally 15, 16, 17, 18, 19, 20, 21, 22, 23, 24, 25, 26, 27, 28, 29, 30, 31 or 32 nucleotides in length, have a GC content of at least 40% and anneal to the target sequence at temperatures of about 54° C. to 72° C. The amplified region may be sequenced as described above, and overlapping sequences assembled into a contiguous sequence. Certain oligonucleotides contemplated by the present invention may, for some embodiments, have lengths of 18, 19, 20, 21, 22, 23, 24, 25, 26, 27, 28, 29, 30, 31, 32, 33-35, 35-40, 41-45, 46-50, 56-60, 61-70, 71-80, 81-90 or more nucleotides.

In general, polypeptides and polynucleotides as described herein are isolated. An “isolated” polypeptide or polynucleotide is one that is removed from its original environment. For example, a naturally occurring protein is isolated if it is separated from some or all of the coexisting materials in the natural system. In certain embodiments, such polypeptides are at least about 90% pure, at least about 95% pure and in certain embodiments, at least about 99% pure. A polynucleotide is considered to be isolated if, for example, it is cloned into a vector that is not a part of the natural environment.

A number of specific siRNA polynucleotide sequences useful for interfering with ILK polypeptide expression are described herein in the Examples and are provided in the Sequence Listing. SiRNA polynucleotides may generally be prepared by any method known in the art, including, for example, solid phase chemical synthesis. Modifications in a polynucleotide sequence may also be introduced using standard mutagenesis techniques, such as oligonucleotide-directed site-specific mutagenesis. Further, siRNAs may be chemically modified or conjugated to improve their serum stability and/or delivery properties as described further herein. Included as an aspect of the invention are the siRNAs described herein wherein the ribose has been removed therefrom. Alternatively, siRNA polynucleotide molecules may be generated by in vitro or in vivo transcription of suitable DNA sequences (e.g., polynucleotide sequences encoding a PTP, or a desired portion thereof), provided that the DNA is incorporated into a vector with a suitable RNA polymerase promoter (such as T7, U6, H1, or SP6). In addition, a siRNA polynucleotide may be administered to a patient, as may be a DNA sequence (e.g., a recombinant nucleic acid construct as provided herein) that supports transcription (and optionally appropriate processing steps) such that a desired siRNA is generated in vivo.

As discussed above, siRNA polynucleotides exhibit desirable stability characteristics and may, but need not, be further designed to resist degradation by endogenous nucleolytic enzymes by using such linkages as phosphorothioate, methylphosphonate, sulfone, sulfate, ketyl, phosphorodithioate, phosphoramidate, phosphate esters, and other such linkages (see, e.g., Agrwal et al., Tetrahedron Lett. 28:3539-3542 (1987); Miller et al., J. Am. Chem. Soc. 93:6657-6665 (1971); Stec et al., Tetrahedron Lett. 26:2191-2194 (1985); Moody et al., Nucleic Acids Res. 12:4769-4782 (1989); Uznanski et al., Nucleic Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein, In: Oligodeoxynucleotides. Antisense Inhibitors of Gene Expression, Cohen, ed., Macmillan Press, London, pp. 97-117 (1989); Jager et al., Biochemistry 27:7237-7246 (1988)).

Any polynucleotide of the invention may be further modified to increase stability in vivo. Possible modifications include, but are not limited to, the addition of flanking sequences at the 5′ and/or 3′ ends; the use of phosphorothioate or 2′O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In certain embodiments, “vectors” mean any nucleic acid- and/or viral-based technique used to deliver a desired nucleic acid.

By “subject” is meant an organism which is a recipient of the nucleic acid molecules of the invention. “Subject” also refers to an organism to which the nucleic acid molecules of the invention can be administered. In certain embodiments, a subject is a mammal or mammalian cells. In further embodiments, a subject is a human or human cells. Subjects of the present invention include, but are not limited to mice, rats, pigs, and non-human primates.

Nucleic acids can be synthesized using protocols known in the art as described in Caruthers et al., 1992, Methods in Enzymology 211, 3-19; Thompson et al., International PCT Publication No. WO 99/54459; Wincott et al., 1995, Nucleic Acids Res. 23, 2677-2684; Wincott et al., 1997, Methods Mol. Bio., 74, 59-68; Brennan et al., 1998, Biotechnol Bioeng., 61, 33-45; and Brennan, U.S. Pat. No. 6,001,311). The synthesis of nucleic acids makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μM scale protocol with a 2.5 min coupling step for 2′-O-methylated nucleotides and a 45 second coupling step for 2′-deoxy nucleotides. Alternatively, syntheses at the 0.2 μM scale can be performed on a 96-well plate synthesizer, such as the instrument produced by Protogene (Palo Alto, Calif.) with minimal modification to the cycle. A 33-fold excess (60 μL of 0.11 M=6.6 μM) of 2′-O-methyl phosphoramidite and a 105-fold excess of S-ethyl tetrazole (60 μL of 0.25 M=15 μM) can be used in each coupling cycle of 2′-O-methyl residues relative to polymer-bound 5′-hydroxyl. A 22-fold excess (40 μL of 0.11 M=4.4 μM) of deoxy phosphoramidite and a 70-fold excess of S-ethyl tetrazole (40 μL of 0.25 M=10 μM) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5 99%. Other oligonucleotide synthesis reagents for the 394 Applied Biosystems, Inc. synthesizer include; detritylation solution is 3% TCA in methylene chloride (ABI); capping is performed with 16% N-methylimidazole in THF (ABI) and 10% acetic anhydride/10% 2,6-lutidine in THF (ABI); and oxidation solution is 16.9 mM 1₂, 49 mM pyridine, 9% water in THF. Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other (see for example, Usman and McSwiggen, supra; Eckstein et al., International PCT Publication No. WO 92/07065; Usman et al., International PCT Publication No. WO 93/15187; Uhlman & Peyman, supra). There are several examples of modified nucleic acid bases known in the art as summarized by Limbach et al., (1994, Nucleic Acids Res. 22, 2183-2196).

Exemplary chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonyhnethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1″ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other (see for example, Usman and McSwiggen, supra; Eckstein et al., International PCT Publication No. WO 92/07065; Usman et al., International PCT Publication No. WO 93/15187; Uhlman & Peyman). There are several examples of modified nucleic acid bases known in the art as summarized by Limbach et al. (1994, Nucleic Acids Res. 22, 2183-2196). Exemplary chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090-14097; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives, and cosmids. Vectors of particular interest include expression vectors, replication vectors, probe generation vectors, and sequencing vectors. In general, a suitable vector contains an origin of replication functional in at least one organism, convenient restriction endonuclease sites, and one or more selectable markers. (See, e.g., WO 01/96584; WO 01/29058; U.S. Pat. No. 6,326,193; U.S. 2002/0007051). Other elements will depend upon the desired use, and will be apparent to those having ordinary skill in the art. For example, the invention contemplates the use of siRNA polynucleotide sequences in the preparation of recombinant nucleic acid constructs including vectors for interfering with the expression of a desired target polypeptide such as a ILK polypeptide in vivo; the invention also contemplates the generation of siRNA transgenic or “knock-out” animals and cells (e.g., cells, cell clones, lines or lineages, or organisms in which expression of one or more desired polypeptides (e.g., a target polypeptide) is fully or partially compromised). An siRNA polynucleotide that is capable of interfering with expression of a desired polypeptide (e.g., a target polypeptide) as provided herein thus includes any siRNA polynucleotide that, when contacted with a subject or biological source as provided herein under conditions and for a time sufficient for target polypeptide expression to take place in the absence of the siRNA polynucleotide, results in a statistically significant decrease (alternatively referred to as “knockdown” of expression) in the level of target polypeptide expression that can be detected. In certain embodiments, the decrease is greater than 10%, 20%, 30%, 40%, 50%, 60%, 70%, 75%, 80%, 85%, 90%, 95% or 98% relative to the expression level of the polypeptide detected in the absence of the siRNA, using conventional methods for determining polypeptide expression as known to the art and provided herein. In certain embodiments, the presence of the siRNA polynucleotide in a cell does not result in or cause any undesired toxic effects, for example, apoptosis or death of a cell in which apoptosis is not a desired effect of RNA interference.

The present invention also relates to vectors and to constructs that include or encode siRNA polynucleotides of the present invention, and in particular to “recombinant nucleic acid constructs” that include any nucleic acids that may be transcribed to yield target polynucleotide-specific siRNA polynucleotides (i.e., siRNA specific for a polynucleotide that encodes a target polypeptide, such as a mRNA) according to the invention as provided above; to host cells which are genetically engineered with vectors and/or constructs of the invention and to the production of siRNA polynucleotides, polypeptides, and/or fusion proteins of the invention, or fragments or variants thereof, by recombinant techniques. SiRNA sequences disclosed herein as RNA polynucleotides may be engineered to produce corresponding DNA sequences using well established methodologies such as those described herein. Thus, for example, a DNA polynucleotide may be generated from any siRNA sequence described herein (including in the Sequence Listing), such that the present siRNA sequences will be recognized as also providing corresponding DNA polynucleotides (and their complements). These DNA polynucleotides are therefore encompassed within the contemplated invention, for example, to be incorporated into the subject invention recombinant nucleic acid constructs from which siRNA may be transcribed.

According to the present invention, a vector may comprise a recombinant nucleic acid construct containing one or more promoters for transcription of an RNA molecule, for example, the human U6 snRNA promoter (see, e.g., Miyagishi et al, Nat. Biotechnol. 20:497-500 (2002); Lee et al., Nat. Biotechnol. 20:500-505 (2002); Paul et al., Nat. Biotechnol. 20:505-508 (2002); Grabarek et al., BioTechniques 34:73544 (2003); see also Sui et al., Proc. Natl. Acad. Sci. USA 99:5515-20 (2002)). Each strand of a siRNA polynucleotide may be transcribed separately each under the direction of a separate promoter and then may hybridize within the cell to form the siRNA polynucleotide duplex. Each strand may also be transcribed from separate vectors (see Lee et al., supra). Alternatively, the sense and antisense sequences specific for a ILK sequence may be transcribed under the control of a single promoter such that the siRNA polynucleotide forms a hairpin molecule (Paul et al., supra). In such an instance, the complementary strands of the siRNA specific sequences are separated by a spacer that comprises at least four nucleotides, but may comprise at least 5, 6, 7, 8, 9, 10, 11, 12, 14, 16, 94 18 nucleotides or more nucleotides as described herein. In addition, siRNAs transcribed under the control of a U6 promoter that form a hairpin may have a stretch of about four uridines at the 3′ end that act as the transcription termination signal (Miyagishi et al., supra; Paul et al., supra). By way of illustration, if the target sequence is 19 nucleotides, the siRNA hairpin polynucleotide (beginning at the 5′ end) has a 19-nucleotide sense sequence followed by a spacer (which as two uridine nucleotides adjacent to the 3′ end of the 19-nucleotide sense sequence), and the spacer is linked to a 19 nucleotide antisense sequence followed by a 4-uridine terminator sequence, which results in an overhang. SiRNA polynucleotides with such overhangs effectively interfere with expression of the target polypeptide (see id.). A recombinant construct may also be prepared using another RNA polymerase III promoter, the H1 RNA promoter, that may be operatively linked to siRNA polynucleotide specific sequences, which may be used for transcription of hairpin structures comprising the siRNA specific sequences or separate transcription of each strand of a siRNA duplex polynucleotide (see, e.g., Brummelkamp et al., Science 296:550-53 (2002); Paddison et al., supra). DNA vectors useful for insertion of sequences for transcription of an siRNA polynucleotide include pSUPER vector (see, e.g., Brummelkamp et al., supra); pAV vectors derived from pCWRSVN (see, e.g., Paul et al., supra); and pIND (see, e.g., Lee et al., supra), or the like.

In certain embodiments, the nucleic acid molecules of the instant invention can be expressed within cells from eukaryotic promoters (e.g., Izant and Weintraub, 1985, Science, 229, 345-352; McGarry and Lindquist, 1986, Proc. Natl. Acad. Sci., USA, 83, 399-403; Scanlon et al., 1991, Proc. Natl. Acad. Sci. USA, 88, 10591-10595; Kashani-Sabet et al., 1992, Antisense Res. Dev., 2, 3-15; Dropulic et al., 1992, J. Virol., 66, 1432-1441; Weerasinghe et al., 1991, J. Virol., 65, 5531-5534; Ojwang et al., 1992, Proc. Natl. Acad. Sci. USA, 89, 10802-10806; Chen et al., 1992, Nucleic Acids Res., 20, 4581-4589; Sarver et al., 1990 Science, 247, 1222-1225; Thompson et al., 1995, Nucleic Acids Res., 23, 2259-2268; Good et al., 1997, Gene Therapy, 4, 45-54). Those skilled in the art will realize that any nucleic acid can be expressed in eukaryotic cells from the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., 1992, Nucleic Acids Symp. Ser., 27, 15-16; Taira et al., 1991, Nucleic Acids Res., 19, 5125-5130; Ventura et al., 1993, Nucleic Acids Res., 21, 3249-3255; Chowrira et al., 1994, J. Biol. Chem., 269, 25856-25864).

In another aspect of the invention, nucleic acid molecules of the present invention, such as RNA molecules, are expressed from transcription units (see for example Couture et al., 1996, TIG., 12, 510-515) inserted into DNA or RNA vectors. The recombinant vectors are preferably DNA plasmids or viral vectors. RNA expressing viral vectors can be constructed based on, but not limited to, adeno-associated virus, retrovirus, adenovirus, lentivirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA and induces RNAi within cell. Delivery of nucleic acid molecule expressing vectors can be systemic, such as by intravenous or intramuscular administration, by administration to target cells ex-planted from the patient or subject followed by reintroduction into the patient or subject, or by any other means that would allow for introduction into the desired target cell (for a review see Couture et al., 1996, TIG., 12, 510-515).

In one aspect, the invention features an expression vector comprising a nucleic acid sequence encoding at least one of the nucleic acid molecules of the instant invention is disclosed. The nucleic acid sequence encoding the nucleic acid molecule of the instant invention is operably linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences may be driven from a promoter for eukaryotic RNA polymerase I (pol l), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-6747; Gao and Huang 1993, Nucleic Acids Res., 21, 2867-2872; Lieber et al., 1993, Methods Enzymol., 217, 47-66; Zhou et al., 1990, Mol. Cell. Biol., 10, 4529-4537). Several investigators have demonstrated that nucleic acid molecules, such as ribozymes expressed from such promoters can function in mammalian cells (e.g., Kashani-Sabet et al., 1992, Antisense Res. Dev., 2, 3-15; Ojwang et al., 1992, Proc. Natl. Acad. Sci. USA, 89, 10802-10806; Chen et al., 1992, Nucleic Acids Res., 20, 4581-4589; Yu et al., 1993, Proc. Natl. Acad. Sci. USA, 90, 6340-6344; L'Huillier et al., 1992, EMBO J., 11, 4411-4418; Lisziewicz et al., 1993, Proc. Natl. Acad. Sci. U.S.A, 90, 8000-8004; Thompson et al., 1995, Nucleic Acids Res., 23, 2259-2268; Sullenger & Cech, 1993, Science, 262, 1566-1569). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830-2836; Noonberg et al., U.S. Pat. No. 5,624,803; Good et al., 1997, Gene Ther., 4, 45-54; Beigelman et al., International PCT Publication No. WO 96/18736). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In yet another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

In another example, the nucleic acids of the invention as described herein (e.g., DNA sequences from which siRNA may be transcribed) herein may be included in any one of a variety of expression vector constructs as a recombinant nucleic acid construct for expressing a target polynucleotide-specific siRNA polynucleotide. Such vectors and constructs include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA, such as vaccinia, adenovirus, fowl pox virus, and pseudorabies. However, any other vector may be used for preparation of a recombinant nucleic acid construct as long as it is replicable and viable in the host.

The appropriate DNA sequence(s) may be inserted into the vector by a variety of procedures. In general, the DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (2001 Molecular Cloning, Third Ed., Cold Spring Harbor Laboratory, Plainview, N.Y.); Maniatis et al. (1982 Molecular Cloning, Cold Spring Harbor Laboratory, Plainview, N.Y.); and elsewhere.

The DNA sequence in the expression vector is operatively linked to at least one appropriate expression control sequences (e.g., a promoter or a regulated promoter) to direct mRNA synthesis. Representative examples of such expression control sequences include LTR or SV40 promoter, the E. coli lac or trp, the phage lambda P_{L} promoter and other promoters known to control expression of genes in prokaryotic or eukaryotic cells or their viruses. Promoter regions can be selected from any desired gene using CAT (chloramphenicol transferase) vectors or other vectors with selectable markers. Two appropriate vectors are pKK232-8 and pCM7. Particular named bacterial promoters include lacl, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a polypeptide (e.g., PTP, MAP kinase kinase, or chemotherapeutic target polypeptide) is described herein.

The expressed recombinant siRNA polynucleotides may be useful in intact host cells; in intact organelles such as cell membranes, intracellular vesicles or other cellular organelles; or in disrupted cell preparations including but not limited to cell homogenates or lysates, microsomes, uni- and multilamellar membrane vesicles or other preparations. Alternatively, expressed recombinant siRNA polynucleotides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Finally, high performance liquid chromatography (HPLC) can be employed for final purification steps.

In certain preferred embodiments of the present invention, the siRNA polynucleotides are detectably labeled, and in certain embodiments the siRNA polynucleotide is capable of generating a radioactive or a fluorescent signal. The siRNA polynucleotide can be detectably labeled by covalently or non-covalently attaching a suitable reporter molecule or moiety, for example a radionuclide such as ³²P (e.g., Pestka et al., 1999 Protein Expr. Purif. 17:203-14), a radiohalogen such as iodine [¹²⁵I or ¹³¹I] (e.g., Wilbur, 1992 Bioconjug. Chem. 3:433-70), or tritium [³H]; an enzyme; or any of various luminescent (e.g., chemiluminescent) or fluorescent materials (e.g., a fluorophore) selected according to the particular fluorescence detection technique to be employed, as known in the art and based upon the present disclosure. Fluorescent reporter moieties and methods for labeling siRNA polynucleotides and/or PTP substrates as provided herein can be found, for example in Haugland (1996 Handbook of Fluorescent Probes and Research Chemicals—Sixth Ed., Molecular Probes, Eugene, Oreg.; 1999 Handbook of Fluorescent Probes and Research Chemicals—Seventh Ed., Molecular Probes, Eugene, Oreg., Internet: http://www.probes.com/lit/) and in references cited therein. Particularly preferred for use as such a fluorophore in the subject invention methods are fluorescein, rhodamine, Texas Red, AlexaFluor-594, AlexaFluor-488, Oregon Green, BODIPY-FL, umbelliferone, dichlorotriazinylamine fluorescein, dansyl chloride, phycoerythrin or Cy-5. Examples of suitable enzymes include, but are not limited to, horseradish peroxidase, biotin, alkaline phosphatase, β-galactosidase and acetylcholinesterase. Appropriate luminescent materials include luminol, and suitable radioactive materials include radioactive phosphorus [³²P]. In certain other preferred embodiments of the present invention, a detectably labeled siRNA polynucleotide comprises a magnetic particle, for example a paramagnetic or a diamagnetic particle or other magnetic particle or the like (preferably a microparticle) known to the art and suitable for the intended use. Without wishing to be limited by theory, according to certain such embodiments there is provided a method for selecting a cell that has bound, adsorbed, absorbed, internalized or otherwise become associated with a siRNA polynucleotide that comprises a magnetic particle.

### Methods of Use and Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar; Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819.

The molecules of the instant invention can be used as pharmaceutical agents. Pharmaceutical agents prevent, inhibit the occurrence, or treat (alleviate a symptom to some extent, in certain embodiments all of the symptoms) of a disease state in a subject.

The negatively charged polynucleotides of the invention can be administered and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A composition or formulation of the siRNA molecules of the present invention refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell. For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes exposes the desired negatively charged nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, DF et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The invention also features the use of the composition comprising surface-modified liposomes containing poly (ethylene glycol) lipids (PEG-modified, branched and unbranched or combinations thereof, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present invention includes nucleic acid compositions, such as siRNA compositions, prepared as described in US 2003/0166601. In this regard, in one embodiment, the present invention provides a composition of the siRNA described herein comprising: 1) a core complex comprising the nucleic acid (e.g., siRNA) and polyethyleneimine; and 2) an outer shell moiety comprising NHS-PEG-VS and a targeting moiety.

Thus, in certain embodiments, siRNA sequences are complexed through electrostatic bonds with a cationic polymer to form a RNAi/nanoplex structure. In certain embodiments, the cationic polymer facilitates cell internalization and endosomal release of its siRNA payload in the cytoplasm of a target cell. Further, in certain embodiments, a hydrophilic steric polymer can be added to the RNAi/cationic polymer nanoplex. In this regard, illustrative steric polymers include a Polyethylene Glycol (PEG) layer. Without being bound by theory, this component helps reduce non-specific tissue interaction, increase circulation time, and minimize immunogenic potential. PEG layers can also enhance siRNA distribution to tumor tissue through the phenomenon of Enhanced Permeability and Retention (EPR) in the often leaky tumor vasculature.

In a further embodiment, the present invention includes nucleic acid compositions prepared for delivery as described in U.S. Pat. Nos. 6,692,911, 7,163,695 and 7,070,807. In this regard, in one embodiment, the present invention provides a nucleic acid of the present invention in a composition comprising poly(Histidine-Lysine) copolymers (HK) (histidine copolymers) as described in U.S. Pat. Nos. 7,163,695, 7,070,807, and 6,692,911 either alone or in combination with PEG (e.g., branched or unbranched PEG or a mixture of both) or in combination with PEG and a targeting moiety. In this regard, in certain embodiments, the present invention provides siRNA molecules in compositions comprising, polylysine, polyhistidine, lysine, histidine, and combinations thereof (e.g., polyhistidine; polyhistidine and polylysine; lysine and polyhistidine; histidine and polylysine; lysine and histidine), gluconic-acid-modified polyhistidine or gluconylated-polyhistidine/transferrin-polylysine. In certain embodiments, the siRNA compositions of the invention comprise branched histidine copolymers (see e.g., U.S. Pat. No. 7,070,807).

In certain embodiments of the present invention a targeting moiety as described above is utilized to target the desired siRNA(s) to a cell of interest. In this regard, as would be recognized by the skilled artisan, targeting ligands are readily interchangeable depending on the disease and siRNA of interest to be delivered. In certain embodiments, the targeting moiety may include an RGD (Arginine, Glycine, Aspartic Acid) peptide ligand that binds to activated integrins on tumor vasculature endothelial cells, such as αvβ3 integrins.

Thus, in certain embodiments, compositions comprising the siRNA molecules of the present invention include at least one targeting moiety, such as a ligand for a cell surface receptor or other cell surface marker that permits highly specific interaction of the composition comprising the siRNA molecule (the “vector”) with the target tissue or cell. More specifically, in one embodiment, the vector preferably will include an unshielded ligand or a shielded ligand. The vector may include two or more targeting moieties, depending on the cell type that is to be targeted. Use of multiple (two or more) targeting moieties can provide additional selectivity in cell targeting, and also can contribute to higher affinity and/or avidity of binding of the vector to the target cell. When more than one targeting moiety is present on the vector, the relative molar ratio of the targeting moieties may be varied to provide optimal targeting efficiency. Methods for optimizing cell binding and selectivity in this fashion are known in the art. The skilled artisan also will recognize that assays for measuring cell selectivity and affinity and efficiency of binding are known in the art and can be used to optimize the nature and quantity of the targeting ligand(s).

A variety of agents that direct compositions to particular cells are known in the art (see, for example, Cotten et al., Methods Enzym, 217: 618, 1993). Illustrative targeting agents include biocompounds, or portions thereof, that interact specifically with individual cells, small groups of cells, or large categories of cells. Examples of useful targeting agents include, but are in no way limited to, low-density lipoproteins (LDLs), transferrin, asiaglycoproteins, gp120 envelope protein of the human immunodeficiency virus (HIV), and diptheria toxin, antibodies, and carbohydrates.

Another example of a targeting moeity is sialyl-Lewis^{x}, where the composition is intended for treating a region of inflammation. Other peptide ligands may be identified using methods such as phage display (F. Bartoli et al., Isolation of peptide ligands for tissue-specific cell surface receptors, in Vector Targeting Strategies for Therapeutic Gene Delivery (Abstracts form Cold Spring Harbor Laboratory 1999 meeting), 1999, p4) and microbial display (Georgiou et al., Ultra-High Affinity Antibodies from Libraries Displayed on the Surface of Microorganisms and Screened by FACS, in Vector Targeting Strategies for Therapeutic Gene Delivery (Abstracts form Cold Spring Harbor Laboratory 1999 meeting), 1999, p 3.). Ligands identified in this manner are suitable for use in the present invention.

Methods have been developed to create novel peptide sequences that elicit strong and selective binding for target tissues and cells such as “DNA Shuffling” (W. P. C. Stremmer, Directed Evolution of Enzymes and Pathways by DNA Shuffling, in Vector Targeting Strategies for Therapeutic Gene Delivery (Abstracts form Cold Spring Harbor Laboratory 1999 meeting), 1999, p.5.) and these novel sequence peptides are suitable ligands for the invention. Other chemical forms for ligands are suitable for the invention such as natural carbohydrates which exist in numerous forms and are a commonly used ligand by cells (Kraling et al., Am. J. Path., 1997, 150, 1307) as well as novel chemical species, some of which may be analogues of natural ligands such as D-amino acids and peptidomimetics and others which are identified through medicinal chemistry techniques such as combinatorial chemistry (P. D. Kassner et al., Ligand Identification via Expression (LIVE.theta.): Direct selection of Targeting Ligands from Combinatorial Libraries, in Vector Targeting Strategies for Therapeutic Gene Delivery (Abstracts form Cold Spring Harbor Laboratory 1999 meeting), 1999, p8.).

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington: The Science and Practice of Pharmacy, 20th Edition. Baltimore, Md.: Lippincott Williams & Wilkins, 2000. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, and in certain embodiments, all of the symptoms of) a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

The nucleic acid compositions of the invention can be used in combination with other nucleic acid compositions that target the same or different areas of the target gene (e.g., ILK), or that target other genes of interest. The nucleic acid compositions of the invention can also be used in combination with any of a variety of treatment modalities, such as chemotherapy, radiation therapy, or small molecule regimens.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.01 mg to about 140 mg per kilogram of body weight per day are useful in the treatment of the disease conditions described herein (about 0.5 mg to about 7 g per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers.

The siRNA molecules of the present invention can be used in a method for treating or preventing an ILK expressing disorder in a subject having or suspected of being at risk for having the disorder, comprising administering to the subject one or more siRNA molecules described herein, thereby treating or preventing the disorder. In this regard, the method provides for treating such diseases described herein, by administering 1, 2, 3, 4, 5, 6, 7, 8, 9, 10, 11, 12, 13, 14, 15 or more siRNA molecules as described herein, such as those provided in SEQ ID NOs:5-118, or a dsRNA thereof.

The present invention provides a method for interfering with expression of a polypeptide, or variant thereof, comprising contacting a subject that comprises at least one cell which is capable of expressing the polypeptide with one or more siRNA polynucleotides as described herein for a time and under conditions sufficient to interfere with expression of the polypeptide.

The nucleic acid molecules of the instant invention, individually, or in combination or in conjunction with other drugs, can be used to treat diseases or conditions associated with altered expression and/or activity of ILK. Thus, the small nucleic acid molecules described herein are useful, for example, in providing compositions to prevent, inhibit, or reduce any one or more of the diseases as described herein and/or other disease states, conditions, or traits associated with ILK gene expression or activity in a subject or organism.

ILK and Angiogenesis: There are several lines of evidences that suggest the important implications for both physiological and pathological angiogenesis. Endothelial ILK plays a critical role in vascular development through integrin-matrix interactions and EC survival. Integrin-mediated outside-in signals cooperate with vascular endothelial growth factor (VEGF) receptor to promote morphological changes, cell proliferation and motility in endothelial cells. Kaneko's group (Kaneko Y. et al. J Cell Sci. 2004; 117:407-415.) demonstrated that VEGF-induced vessel morphogenesis of human umbilical vein endothelial cells (HUVEC) was inhibited by the transfection of a dominant negative, kinase-deficient ILK (ILK-KD), as well as by treatment with the PI 3-K inhibitor LY294002. VEGF induced phosphorylation of PKB/Akt in an ILK-dependent manner. Furthermore, transfection of antisense ILK (ILK-AS) blocked the survival effect of VEGF. VEGF-mediated decrease in caspase activity was reversed by both ILK-KD and ILK-AS. In addition, migration and proliferation of HUVEC induced by VEGF were suppressed by the inhibition of ILK.

Friedrich and coworkers (Friedrich EB et al. Mol. Cell Biol. 2004; 24: 8134-8144.) showed that endothelial cell (EC)-specific deletion of ILK in mice confers placental insufficiency with decreased labyrinthine vascularization, yielding no viable offspring. Deletion of ILK in zebra fish using antisense morpholino oligonucleotides results in marked patterning abnormalities of the vasculature and is similarly lethal. Ex vivo deletion of ILK from purified EC of adult mice indicated downregulation of the active-conformation of betal integrins with a striking increase in EC apoptosis. There was also reduced phosphorylation of the ILK kinase substrate, Akt. However, phenotypic rescue of ILK-deficient EC by wild-type ILK, but not by a constitutively active mutant of Akt, suggests regulation of EC survival by ILK in an Akt-independent manner.

Neovascularization is a dynamic process of detachment and reattachment of ECs and endothelial progenitor cells (EPCs). A team led by Kim (Cho H J et al. Arterioscler Thromb Vasc Biol. 2005, 25:1154-1160.) discovered that ILK expression in ECs and EPCs was decreased in various stress conditions, and the gene transfer of ILK protected ECs and EPCs from temporary anchorage or nutrient deprivation. ILK overexpression protects ECs and EPCs from anchorage- or nutrient-deprived stress and enhances neovascularization. Furthermore, ILK gene transfer in EPCs significantly enhanced neovascularization in vivo. Recently, the same group (Lee SP. Et al. Circulation. 2006 114:150-159.) reported that ILK responds to hypoxia in ECs and regulates the expression of stromal cell-derived factor-1 (SDF-1) and intercellular adhesion molecule-1 (ICAM-1) through nuclear factor-kappaB and hypoxia-inducible factor-1 alpha and induces recruitment of EPCs to ischemic areas. Blockade of ILK in hypoxic ECs significantly abrogated the expression of both molecules, which led to decreased EPC incorporation into ECs. Overexpression of ILK in the Matrigel plug significantly improved neovascularization in vivo, whereas the blockade of ILK resulted in the opposite effect.

Thus, the compositions of the present invention can be used in the treatment of disease associated with abnormal or pathological angiogenesis, such as, but not limited to, any of a variety of cancers, psoriasis and age-related macular degeneration.

The nucleic acid molecules of the instant invention, individually, or in combination or in conjunction with other drugs, can also be used to prevent diseases or conditions associated with altered activity and/or expression of ILK in individuals that are suspected of being at risk for developing such a disease or condition. For example, to treat or prevent a disease or condition associated with the expression levels of ILK, the subject having the disease or condition, or suspected of being at risk for developing the disease or condition, can be treated, or other appropriate cells can be treated, as is evident to those skilled in the art, individually or in combination with one or more drugs under conditions suitable for the treatment. Thus, the present invention provides methods for treating or preventing diseases or conditions which respond to the modulation of ILK expression comprising administering to a subject in need thereof an effective amount of a composition comprising one or more of the nucleic acid molecules of the invention, such as those set forth in SEQ ID NOs:5-118. In one embodiment, the present invention provides methods for treating or preventing diseases associated with expression of ILK comprising administering to a subject in need thereof an effective amount of any one or more of the nucleic acid molecules of the invention, such as those provided in SEQ ID NOs:5-118, such that the expression of ILK in the subject is down-regulated, thereby treating or preventing the disease associated with expression of ILK. Thus, the present invention provides methods for treating or preventing diseases or conditions which respond to the modulation of ILK expression comprising administering to a subject in need thereof an effective amount of a composition comprising one or more of the nucleic acid molecules of the invention. In one embodiment, the present invention provides methods for treating or preventing diseases associated with expression of ILK comprising administering to a subject in need thereof an effective amount of any one or more of the nucleic acid molecules of the invention such that the expression of ILK in the subject is modulated, thereby treating or preventing the disease associated with expression of ILK. In this regard, the compositions of the invention can be used in methods for treating or preventing brain, esophageal, bladder, cervical, breast, lung, prostate, colorectal, pancreatic, head and neck, prostate, thyroid, kidney, and ovarian cancer, melanoma, lymphoma, glioma, glioblastoma, multidrug resistant cancers, and any other cancerous diseases, cardiac disorders (e.g., cardiomyopathy, cardiovascular disease, congenital heart disease, coronary heart disease, heart failure, hypertensive heart disease, inflammatory heart disease, valvular heart disease), or other conditions which respond to the modulation of ILK expression. The compositions of the invention can also be used in methods for treating any of a number of known metabolic disorders including inherited metabolic disorders. Metabolic disorders that may be treated include, but are not limited to diabetes mellitus, hyperlipidemia, lactic acidosis, phenylketonuria, tyrosinemias, alcaptonurta, isovaleric acidemia, homocystinuria, urea cycle disorders, or an organic acid metabolic disorder, propionic acidemia, methylmalonic acidemia, glutaric aciduria Type 1, acid lipase disease, amyloidosis, Barth syndrome, biotinidase deficiency (BD), carnitine palitoyl transferase deficiency type II (CPT-II), central pontine myelinolysis, muscular dystrophy, Farber's disease, G6PD deficiency (Glucose-6-Phosphate Dehydrogenase), gangliosidoses, trimethylaminuria, Lesch-Nyhan syndrome, lipid storage diseases, metabolic myopathies, methylmalonic aciduria (MMA), mitochondrial myopathies, MPS (Mucopolysaccharidoses) and related diseases, mucolipidoses, mucopolysaccharidoses, multiple CoA carboxylase deficiency (MCCD), nonketotic hyperglycinemia, Pompe disease, propionic acidemia (PROP), and Type I glycogen storage disease.

ILK in Inflammation: Leukocyte extravasation is an important step of inflammation, in which integrins have been demonstrated to play an essential role by mediating the interaction of leukocytes with the vascular endothelium and the subendothelial extracellular matrix. As a linkage between integrin and the cytoskeleton system, ILK is a critical molecule involved in the cell-cell, and cell-matrix interaction. ILK is highly expressed in human mononuclear leukocyte subsets and is activated by exposure of leukocytes to chemokines, such as MCP-1 (Friedrich EB et al. J Biol. Chem; 2002, 19: 16371-16375). It may play a role in the sequential modulation of integrin affinity and avidity, which has been proposed as a crucial mechanism to facilitate leukocyte adhesion and transendothelial migration. Chemokine-triggered activation is sustained for several minutes and is dependent on PI3K. Interestingly, overexpression of ILK in human monocytic cells diminishes β₁ integrin/VCAM-1-dependent firm adhesion to human vascular endothelial cells. These data implicate ILK in the dynamic signaling events involved in the regulation of leukocyte integrin avidity for endothelial substrates. Recently, Yoshimi and coworkers (Yoshimi R. et al., J. Immunol, 2006, 176: 3611-3624.) dominstrated that an ILK-binding protein, gamma-parvin, could form a complex with some important cytoskeletal proteins. RNAi-mediated knock-down of parvin inhibits attachment and spreading of inflammatory cells on matrix protein-coated surface. ILK-parvin complex is critically involved in the initial integrin signaling for leukocyte migration.

Thus, the compositions of the invention can be used in methods for preventing inflammatory diseases in individuals suspected of being at risk for developing them, and methods for treating inflammatory diseases, such as, but not limited to, asthma, Chronic Obstructive Pulmonary Disease (COPD), inflammatory bowel disease, ankylosing spondylitis, Reiter's syndrome, Crohn's disease, ulcerative colitis, systemic lupus erythematosus, psoriasis, artherosclerosis, rheumatoid arthritis, osteoarthritis, or multiple sclerosis. The compositions of the invention can also be used in methods for reducing inflammation.

In a further embodiment, the nucleic acid molecules of the invention, such as isolated siRNA, can be used in combination with other known treatments to treat conditions or diseases discussed herein. For example, the described molecules can be used in combination with one or more known therapeutic agents to treat the diseases as described herein or other conditions which respond to the modulation of ILK expression. Such treatments include, but are not limited to chemotherapy, radiation therapy, or small molecule regimens, and also include, but are not limited to, such drugs as beta-blockers (e.g., Acebutolol (Sectral); Atenolol (Tenormin); Betaxolol (Kerlone); Bisoprolol (Zebeta); Carvedilol (Coreg); Labetalol (Normodyne, Trandate); Metoprolol succinate (long acting Toprol XL); Metoprolol tartrate (Lopressor); Nadolol (Corgard); Penbutolol (Levatol); Pindolol (Visken); Propranolol (Inderal); Propranolol long-acting (Betachron, Inderal-LA, Innopran XL); Timolol (Blocadren)) and/or antioxidants (such as, but not limited to Alpha Lipoic Acid, Beta-carotene, Ubiquinone, Cucurmin, Cysteine, Glutathione, Oligomeric Proanthocyanidins, Pychnogenol, Selenium, Vitamin A, C, E, Zinc), immunosuppressants (such as, but not limited to, azathioprine, basiliximab, daclizumab, sirolimus, tacrolimus, muromonab-CD3, cyclophosphamide, mycophenolate, cyclosporine, methotrexate and mercaptopurine), anticoagulants (e.g., heparin; warfarin), antiplatelets (e.g., aspirin; clopidogrel; dipyridamole; ticlopidine).

Compositions and methods are known in the art for identifying subjects having, or suspected of being at risk for having the diseases or disorders associated with expression of ILK as described herein.

### Examples

### Example 1

### SiRNA Molecules Inhibit Human ILK Expression

Human ILK siRNA molecules were designed using the publicly available sequences for the human ILK gene as set forth in GENBANK accession numbers: U40282.1 (SEQ ID NO:1); NM_{—}004517.2 (SEQ ID NO:2); NM_{—}001014794.1 (SEQ ID NO:3); NM_{—}001014795.1 (SEQ ID NO:4). Alignments showed no mismatch in the coding region of these four sequences. Corresponding amino acid sequences are set forth in SEQ ID NOs:119-122, respectively.

Candidate siRNA molecules were synthesized using standard techniques. siRNA candidates are shown in Table 1.

TABLE 1
ILK siRNA Candidates

| | | | SEQ |
|---|---|---|---|
| Start | Sequence | | ID |
| Position | (Sense-strand/antisense-strand) | GC% | NO: |
| 221 | 5′-r(CAGCCAGUCAUGGACACCGUGAUAU) -3′ | 52 | 5 |
| | 3′-( GUCGGUCAGUACCUGUGGCACUAUA)r-5′ | | 6 |
| 225 | 5′-r(CAGUCAUGGACACCGUGAUAUUGUA) -3′ | 44 | 7 |
| | 3′- (GUCAGUACCUGUGGCACUAUAACAU)r-5′ | | 8 |
| 272 | 5′-r(CAGACAUCAAUGCAGUGAAUGAACA) -3′ | 40 | 9 |
| | 3′- (GUCUGUAGUUACGUCACUUACUUGU)r-5′ | | 10 |
| 455 | 5′-r(CAGAGAAGAUGGGCCAGAAUCUCAA) -3′ | 48 | 11 |
| | 3′- (GUCUCUUCUACCCGGUCUUAGAGUU)r-5′ | | 12 |
| 467 | 5′-r(GCCAGAAUCUCAACCGUAUUCCAUA) -3′ | 44 | 13 |
| | 3′- (CGGUCUUAGAGUUGGCAUAAGGUAU)r-5′ | | 14 |
| 560 | 5′-r(GCAUUGACUUCAAACAGCUUAACUU) -3′ | 36 | 15 |
| | 3′- (CGUAACUGAAGUUUGUCGAAUUGAA)r-5′ | | 16 |
| 824 | 5′-r(GAUCCCUCUACAAUGUACUACAUGA) -3′ | 40 | 17 |
| | 3′- (CUAGGGAGAUGUUACAUGAUGUACU)r-5′ | | 18 |
| 1165 | 5′-r(GAGGUACCCUUUGCUGACCUCUCCA) -3′ | 56 | 19 |
| | 3′- (CUCCAUGGGAAACGACUGGAGAGGU)r-5′ | | 20 |
| 1197 | 5′-r(GAUUGGAAUGAAGGUGGCAUUGGAA) -3′ | 44 | 21 |
| | 3′- (CUAACCUUACUUCCACCGUAACCUU)r-5′ | | 22 |
| 1255 | 5′-r(CCUCAUGUGUGUAAGCUCAUGAAGA) -3′ | 44 | 23 |
| | 3′- (GGAAUACACACAUUGCAGUACUUCU)r-5′ | | 24 |
| 137 | 5′-r(GCUCUGCUGUGGUUGAGAUGUUGAU) -3′ | 48 | 25 |
| | 3′- (CGAGACGACACCAACUCUACAACUA)r-5′ | | 26 |
| 139 | 5′-r(UCUGCUGUGGUUGAGAUGUUGAUCA) -3′ | 44 | 27 |
| | 3′- ( AGACGACACCAACUCUACAACUAGU)r-5′ | | 28 |
| 224 | 5′-r(CCAGUCAUGGACACCGUGAUAUUGU) -3′ | 48 | 29 |
| | 3′- (GGUCAGUACCUGUGGCACUAUAACA)r-5′ | | 30 |
| 235 | 5′-r(CACCGUGAUAUUGUACAGAAGCUAU) -3′ | 40 | 31 |
| | 3′- (GUGGCACUAUAACAUGUCUUCGAUA)r-5′ | | 32 |
| 236 | 5′-r(ACCGUGAUAUUGUACAGAAGCUAUU) -3′ | 36 | 33 |
| | 3′- (UGGCACUAUAACAUGUCUUCGAUAA)r-5′ | | 34 |
| 260 | 5′-r(CAGAAGCUAUUGCAGUACAAGGCAG) -3′ | 48 | 35 |
| | 3′- (GUCUUCGAUAACGUCAUGUUCCGUC)r-5′ | | 36 |
| 262 | 5′-r(CAGUACAAGGCAGACAUCAAUGCAG) -3′ | 48 | 37 |
| | 3′- (GUCAUGUUCCGUCUGUAGUUACGUC)r-5′ | | 38 |
| 267 | 5′-r(CAAGGCAGACAUCAAUGCAGUGAAU) -3′ | 44 | 39 |
| | 3′- (GUUCCGUCUGUAGUUACGUCACUUA)r-5′ | | 40 |
| 278 | 5′-r(UCAAUGCAGUGAAUGAACACGGGAA) -3′ | 44 | 41 |
| | 3′- (AGUUACGUCACUUACUUGUGCCCUU)r-5′ | | 42 |
| 371 | 5′-r(CCCUUGUCAGCAUCUGUAACAAGUA) -3′ | 44 | 43 |
| | 3′- (GGGAACAGUCGUAGACAUUGUUCAU)r-5′ | | 44 |
| 549 | 5′-r(CAAACACUCUGGCAUUGACUUCAAA) -3′ | 40 | 45 |
| | 3′- (GUUUGUGAGACCGUAACUGAAGUUU)r-5′ | | 46 |
| 569 | 5′-r(UCAAACAGCUUAACUUCCUGACGAA) -3′ | 40 | 47 |
| | 3′- (AGUUUGUCGAAUUGAAGGACUGCUU)r-5′ | | 48 |
| 591 | 5′-r(GAAGCUCAACGAGAAUCACUCUGGA) -3′ | 48 | 49 |
| | 3′- (CUUCGAGUUGCUCUUAGUGAGACCU)r-5′ | | 50 |
| 597 | 5′-r(CAACGAGAAUCACUCUGGAGAGCUA) -3′ | 48 | 51 |
| | 3′- (GUUGCUCUUAGUGAGACCUCUCGAU)r-5′ | | 52 |
| 601 | 5′-r(GAGAAUCACUCUGGAGAGCUAUGGA) -3′ | 48 | 53 |
| | 3′- (CUCUUAGUGAGACCUCUCGAUACCU)r-5′ | | 54 |
| 602 | 5′-r(AGAAUCACUCUGGAGAGCUAUGGAA) -3′ | 44 | 55 |
| | 3′- (UCUUAGUGAGACCUCUCGAUACCUU)r-5′ | | 56 |
| 635 | 5′-r(GGCAGGGCAAUGACAUUGUCGUGAA) -3′ | 52 | 57 |
| | 3′- (CCGUCCCGUUACUGUAACAGCACUU)r-5′ | | 58 |
| 641 | 5′-r(GCAAUGACAUUGUCGUGAAGGUGCU) -3′ | 48 | 59 |
| | 3′- (CGUUACUGUAACAGCACUUCCACGA)r-5′ | | 60 |
| 689 | 5′-r(GGAAGAGCAGGGACUUCAAUGAAGA) -3′ | 48 | 61 |
| | 3′- (CCUUCUCGUCCCUGAAGUUACUUCU)r-5′ | | 62 |
| 779 | 5′-r(CACCUGCUCCUCAUCCUACUCUCAU) -3′ | 52 | 63 |
| | 3′- (GUGGACGAGGAGUAGGAUGAGAGUA)r-5′ | | 64 |
| 811 | 5′-r(UGGAUGCCGUAUGGAUCCCUCUACA) -3′ | 52 | 65 |
| | 3′- (ACCUACGGCAUACCUAGGGAGAUGU)r-5′ | | 66 |
| 871 | 5′-r(CAGAGCCAGGCUGUGAAGUUUGCUU) -3′ | 52 | 67 |
| | 3′- (GUCUCGGUCCGACACUUCAAACGAA)r-5′ | | 68 |
| 875 | 5′-r(GCCAGGCUGUGAAGUUUGCUUUGGA) -3′ | 52 | 69 |
| | 3′- (CGGUCCGACACUUCAAACGAAACCU)r-5′ | | 70 |
| 939 | 5′-r(CCUCAUCCCACGACAUGCACUCAAU) -3′ | 52 | 71 |
| | 3′- (GGAGUAGGGUGCUGUACGUGAGUUA)r-5′ | | 72 |
| 959 | 5′-r(UCAAUAGCCGUAGUGUAAUGAUUGA) -3′ | 36 | 73 |
| | 3′- (AGUUAUCGGCAUCACAUUACUAACU)r-5′ | | 74 |
| 981 | 5′-r(UGAUGAGGACAUGACUGCCCGAAUU) -3′ | 48 | 75 |
| | 3′- (ACUACUCCUGUACUGACGGGCUUAA)r-5′ | | 76 |
| 985 | 5′-r(GAGGACAUGACUGCCCGAAUUAGCA) -3′ | 52 | 77 |
| | 3′- (CUCCUGUACUGACGGGCUUAAUCGU)r-5′ | | 78 |
| 998 | 5′-r(CCCGAAUUAGCAUGGCUGAUGUCAA) -3′ | 48 | 79 |
| | 3′- (GGGCUUAAUCGUACCGACUACAGUU)r-5′ | | 80 |
| 1006 | 5′-r(AGCAUGGCUGAUGUCAAGUUCUCUU) -3′ | 44 | 81 |
| | 3′- (UCGUACCGACUACAGUUCAAGAGAA)r-5′ | | 82 |
| 1010 | 5′-r(UGGCUGAUGUCAAGUUCUCUUUCCA) -3′ | 44 | 83 |
| | 3′- (ACCGACUACAGUUCAAGAGAAAGGU)r-5′ | | 84 |
| 1011 | 5′-r(GGCUGAUGUCAAGUUCUCUUUCCAA) -3′ | 44 | 85 |
| | 3′- (CCGACUACAGUUCAAGAGAAAGGUU)r-5′ | | 86 |
| 1074 | 5′-r(CGAAGCUCUGCAGAAGAAGCCUGAA) -3′ | 52 | 87 |
| | 3′- (GCUUCGAGACGUCUUCUUCGGACUU)r-5′ | | 88 |
| 1084 | 5′-r(CAGAAGAAGCCUGAAGACACAAACA) -3′ | 44 | 89 |
| | 3′- (GUCUUCUUCGGACUUCUGUGUUUGU)r-5′ | | 90 |
| 1181 | 5′-r(ACCUCUCCAAUAUGGAGAUUGGAAU) -3′ | 40 | 91 |
| | 3′- (UGGAGAGGUUAUACCUCUAACCUUA)r-5′ | | 92 |
| 1271 | 5′-r(UCAUGAAGAUCUGCAUGAAUGAAGA) -3′ | 36 | 93 |
| | 3′- (AGUACUUCUAGACGUACUUACUUCU)r-5′ | | 94 |
| 1280 | 5′-r(UCUGCAUGAAUGAAGACCCUGCAAA) -3′ | 44 | 95 |
| | 3′- (AGACGUACUUACUUCUGGGACGUUU)r-5′ | | 96 |
| 1291 | 5′-r(GAAGACCCUGCAAAGCGACCCAAAU) -3′ | 52 | 97 |
| | 3′- (CUUCUGGGACGUUUCGCUGGGUUUA)r-5′ | | 98 |
| 1292 | 5′-r(AAGACCCUGCAAAGCGACCCAAAUU) -3′ | 48 | 99 |
| | 3′- (UUCUGGGACGUUUCGCUGGGUUUAA)r-5′ | | 100 |
| 1293 | 5′-r(AGACCCUGCAAAGCGACCCAAAUUU) -3′ | 48 | 101 |
| | 3′- (UCUGGGACGUUUCGCUGGGUUUAAA)r-5′ | | 102 |
| 1301 | 5′-r(CAAAGCGACCCAAAUUUGACAUGAU) -3′ | 40 | 103 |
| | 3′- (GUUUCGCUGGGUUUAAACUGUACUA)r-5′ | | 104 |
| 1302 | 5′-r(AAAGCGACCCAAAUUUGACAUGAUU) -3′ | 36 | 105 |
| | 3′- (GUUUCGCUGGGUUUAAACUGUACUA)r-5′ | | 106 |
| 1349 | 5′-r(AGGACAAGUAGGACUGGAAGGUCCU) -3′ | 52 | 107 |
| | 3′- (UCCUGUUCAUCCUGACCUUCCAGGA)r-5′ | | 108 |
| 1357 | 5′-r(UAGGACUGGAAGGUCCUUGCCUGAA) -3′ | 52 | 109 |
| | 3′- (AUCCUGACCUUCCAGGAACGGACUU)r-5′ | | 110 |
| 1530 | 5′-r(GCUCAGAGCUUUGUCACUUGCCACA) -3′ | 52 | 111 |
| | 3′- (CGAGUCUCGAAACAGUGAACGGUGU)r-5′ | | 112 |
| 1551 | 5′-r(CACAUGGUGUCUUCCAACAUGGGAG) -3′ | 52 | 113 |
| | 3′- (GUGUACCACAGAAGGUUGUACCCUC)r-5′ | | 114 |
| 1584 | 5′-r(CCCGCCUGUCACAAUAAAGUUUAUU) -3′ | 40 | 115 |
| | 3′- (GGGCGGACAGUGUUAUUUCAAAUAA)r-5′ | | 116 |
| 1585 | 5′-r(CCGCCUGUCACAAUAAAGUUUAUUA) -3′ | 36 | 117 |
| | 3′- (GGCGGACAGUGUUAUUUCAAAUAAU)r-5′ | | 118 |

The above siRNA molecules are useful for modulating the expression of ILK and are useful in a variety of therapeutic settings, for example, in the treatment of a number of cancers, inflammatory diseases and other diseases as described herein.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An isolated small interfering RNA (siRNA) polynucleotide, comprising at least one nucleotide sequence selected from the group consisting of SEQ ID NOs:5-118.

2. The siRNA polynucleotide of claim 1 that comprises at least one nucleotide sequence selected from the group consisting of SEQ ID NOs:5-118 and the complementary polynucleotide thereto.

3. The small interfering RNA polynucleotide of claim 1 that inhibits expression of a ILK polypeptide, wherein the ILK polypeptide comprises an amino acid sequence as set forth in SEQ ID NOs:119-122, or that is encoded by the polynucleotide as set forth in any one of SEQ ID NOS:1-4.

4. The siRNA polynucleotide of claim 1 wherein the nucleotide sequence of the siRNA polynucleotide differs by one, two, three or four nucleotides at any position of a sequence selected from the group consisting of the sequences set forth in SEQ ID NOS: 5-118, or the complement thereof.

5. The siRNA polynucleotide of claim 2 wherein the nucleotide sequence of the siRNA polynucleotide differs by at least one mismatched base pair between a 5′ end of an antisense strand and a 3′ end of a sense strand of a sequence selected from the group consisting of the sequences set forth in SEQ ID NOS:5-118.

6. The siRNA polynucleotide of claim 5 wherein the mismatched base pair is selected from the group consisting of G:A, C:A, C:U, G:G, A:A, C:C, U:U, C:T, and U:T.

7. The siRNA polynucleotide of claim 5 wherein the mismatched base pair comprises a wobble base pair (G:U) between the 5′ end of the antisense strand and the 3′ end of the sense strand.

8. The siRNA polynucleotide of claim 1 wherein the polynucleotide comprises at least one synthetic nucleotide analogue of a naturally occurring nucleotide.

9. The siRNA polynucleotide of claim 1 wherein the polynucleotide is linked to a detectable label.

10. The siRNA polynucleotide of claim 9 wherein the detectable label is a reporter molecule.

11. The siRNA of claim 10 wherein the reporter molecule is selected from the group consisting of a dye, a radionuclide, a luminescent group, a fluorescent group, and biotin.

12. The siRNA polynucleotide of claim 11 wherein the detectable label is a magnetic particle.

13. An isolated siRNA molecule that inhibits expression of a ILK gene, wherein the siRNA molecule comprises a nucleic acid that targets the sequence provided in SEQ ID NOs:1-4, or a variant thereof wherein the variant exhibits kinase activity.

14. The siRNA molecule of claim 13, wherein the siRNA comprises any one of the single stranded RNA sequences provided in SEQ ID NOs:5-118, or a double-stranded RNA thereof.

15. The siRNA molecule of claim 14 wherein the siRNA molecule down regulates expression of an ILK gene via RNA interference (RNAi).

16. A composition comprising one or more of the siRNA polynucleotides of claim 1, and a physiologically acceptable carrier.

17. The composition of claim 16 wherein the composition comprises a positively charged polypeptide.

18. The composition of claim 17 wherein the positively charged polypeptide comprises poly(Histidine-Lysine).

19. The composition of claim 16 further comprising a targeting moiety.

20. A method for treating or preventing a cancer in a subject having or suspected of being at risk for having the cancer, comprising administering to the subject the composition of claim 16, thereby treating or preventing the cancer.

21. A method for inhibiting the synthesis or expression of ILK comprising contacting a cell expressing ILK with any one or more siRNA molecules wherein the one or more siRNA molecules comprises a sequence selected from the sequences provided in SEQ ID NOs:5-118, or a double-stranded RNA thereof.

22. The method of claim 21 wherein a nucleic acid sequence encoding ILK comprises the sequence set forth in any one of SEQ ID NOS:1-4.

23. A method for reducing the severity of a cancer in a subject, comprising administering to the subject the composition of claim 16, thereby reducing the severity of the cancer.

24. A recombinant nucleic acid construct comprising a nucleic acid that is capable of directing transcription of a small interfering RNA (siRNA), the nucleic acid comprising:
(a) a first promoter; (b) a second promoter; and (c) at least one DNA polynucleotide segment comprising at least one polynucleotide that is selected from the group consisting of (i) a polynucleotide comprising the nucleotide sequence set forth in any one of SEQ ID NOs:5-118, and (ii) a polynucleotide of at least 18 nucleotides that is complementary to the polynucleotide of (i), wherein the DNA polynucleotide segment is operably linked to at least one of the first and second promoters, and wherein the promoters are oriented to direct transcription of the DNA polynucleotide segment and of the complement thereto.

25. The recombinant nucleic acid construct of claim 24, comprising at least one enhancer that is selected from a first enhancer operably linked to the first promoter and a second enhancer operably linked to the second promoter.

26. The recombinant nucleic acid construct of claim 24, comprising at least one transcriptional terminator that is selected from (i) a first transcriptional terminator that is positioned in the construct to terminate transcription directed by the first promoter and (ii) a second transcriptional terminator that is positioned in the construct to terminate transcription directed by the second promoter.

27. An isolated host cell transformed or transfected with the recombinant nucleic acid construct according to claim 24.


SEQUENCE LISTING (122 sequences)

SEQ ID NO: 1 - DNA, 1789 bp, Homo sapiens
gaattcatctgtcgactgctaccacgggagttccccggagaaggatcctgcagcccgagtcccgaggataaagcttggggttcatcctccttccctggatcactccacagtcctcaggcttccccaatccaggggactcggcgccgggacgctgctatggacgacattttcactcagtgccgggagggcaacgcagtcgccgttcgcctgtggctggacaacacggagaacgacctcaaccagggggacgatcatggcttctcccccttgcactgggcctgccgagagggccgctctgctgtggttgagatgttgatcatgcggggggcacggatcaatgtaatgaaccgtggggatgacacccccctgcatctggcagccagtcatggacaccgtgatattgtacagaagctattgcagtacaaggcagacatcaatgcagtgaatgaacacgggaatgtgcccctgcactatgcctgtttttggggccaagatcaagtggcagaggacctggtggcaaatggggcccttgtcagcatctgtaacaagtatggagagatgcctgtggacaaagccaaggcacccctgagagagcttctccgagagcgggcagagaagatgggccagaatctcaaccgtattccatacaaggacacattctggaaggggaccacccgcactcggccccgaaatggaaccctgaacaaacactctggcattgacttcaaacagcttaacttcctgacgaagctcaacgagaatcactctggagagctatggaagggccgctggcagggcaatgacattgtcgtgaaggtgctgaaggttcgagactggagtacaaggaagagcagggacttcaatgaagagtgtccccggctcaggattttctcgcatccaaatgtgctcccagtgctaggtgcctgccagtctccacctgctcctcatcctactctcatcacacactggatgccgtatggatccctctacaatgtactacatgaaggcaccaatttcgtcgtggaccagagccaggctgtgaagtttgctttggacatggcaaggggcatggccttcctacacacactagagcccctcatcccacgacatgcactcaatagccgtagtgtaatgattgatgaggacatgactgcccgaattagcatggctgatgtcaagttctctttccaatgtcctggtcgcatgtatgcacctgcctgggtagcccccgaagctctgcagaagaagcctgaagacacaaacagacgctcagcagacatgtggagttttgcagtgcttctgtgggaactggtgacacgggaggtaccctttgctgacctctccaatatggagattggaatgaaggtggcattggaaggccttcggcctaccatcccaccaggtatttcccctcatgtgtgtaagctcatgaagatctgcatgaatgaagaccctgcaaagcgacccaaatttgacatgattgtgcctatccttgagaagatgcaggacaagtaggactggaaggtccttgcctgaactccagaggtgtcgggacatggttgggggaatgcacctccccaaagcagcaggcctctggttgcctcccccgcctccagtcatggtactaccccagcctggggtccatccccttcccccatccctaccactgtgcgcaagaggggcgggctcagagctttgtcacttgccacatggtgtcttccaacatgggagggatcagccccgcctgtcacaataaagtttattatgaaaaaaaaaaaaaaaaaaaaaa

SEQ ID NO: 2 - DNA, 1817 bp, Homo sapiens
agcgccacgggcggggcggggccggcggcgggctgcgggcgcggccggacgggagttccccggagaaggatcctgcagcccgagtcccgaggataaagcttggggttcatcctccttccctggatcactccacagtcctcaggcttccccaatccaggggactcggcgccgggacgctgctatggacgacattttcactcagtgccgggagggcaacgcagtcgccgttcgcctgtggctggacaacacggagaacgacctcaaccagggggacgatcatggcttctcccccttgcactgggcctgccgagagggccgctctgctgtggttgagatgttgatcatgcggggggcacggatcaatgtaatgaaccgtggggatgacacccccctgcatctggcagccagtcatggacaccgtgatattgtacagaagctattgcagtacaaggcagacatcaatgcagtgaatgaacacgggaatgtgcccctgcactatgcctgtttttggggccaagatcaagtggcagaggacctggtggcaaatggggcccttgtcagcatctgtaacaagtatggagagatgcctgtggacaaagccaaggcacccctgagagagcttctccgagagcgggcagagaagatgggccagaatctcaaccgtattccatacaaggacacattctggaaggggaccacccgcactcggccccgaaatggaaccctgaacaaacactctggcattgacttcaaacagcttaacttcctgacgaagctcaacgagaatcactctggagagctatggaagggccgctggcagggcaatgacattgtcgtgaaggtgctgaaggttcgagactggagtacaaggaagagcagggacttcaatgaagagtgtccccggctcaggattttctcgcatccaaatgtgctcccagtgctaggtgcctgccagtctccacctgctcctcatcctactctcatcacacactggatgccgtatggatccctctacaatgtactacatgaaggcaccaatttcgtcgtggaccagagccaggctgtgaagtttgctttggacatggcaaggggcatggccttcctacacacactagagcccctcatcccacgacatgcactcaatagccgtagtgtaatgattgatgaggacatgactgcccgaattagcatggctgatgtcaagttctctttccaatgtcctggtcgcatgtatgcacctgcctgggtagcccccgaagctctgcagaagaagcctgaagacacaaacagacgctcagcagacatgtggagttttgcagtgcttctgtgggaactggtgacacgggaggtaccctttgctgacctctccaatatggagattggaatgaaggtggcattggaaggccttcggcctaccatcccaccaggtatttcccctcatgtgtgtaagctcatgaagatctgcatgaatgaagaccctgcaaagcgacccaaatttgacatgattgtgcctatccttgagaagatgcaggacaagtaggactggaaggtccttgcctgaactccagaggtgtcgggacatggttgggggaatgcacctccccaaagcagcaggcctctggttgcctcccccgcctccagtcatggtactaccccagccatggggtccatccccttcccccatccctaccactgtggccccaagaggggcgggctcagagctttgtcacttgccacatggtgtctcccaacatgggagggatcagccccgcctgtcacaataaagtttattatgaaaaaaaaaaaaaaaaaaaaaa

SEQ ID NO: 3 - DNA, 1771 bp, Homo sapiens
agcgccacgggcggggcggggccggcggcgggctgcgggcgcggccggacgggagttccccggagaaggatcctgcagcccgagtcccgtcctcaggcttccccaatccaggggactcggcgccgggacgctgctatggacgacattttcactcagtgccgggagggcaacgcagtcgccgttcgcctgtggctggacaacacggagaacgacctcaaccagggggacgatcatggcttctcccccttgcactgggcctgccgagagggccgctctgctgtggttgagatgttgatcatgcggggggcacggatcaatgtaatgaaccgtggggatgacacccccctgcatctggcagccagtcatggacaccgtgatattgtacagaagctattgcagtacaaggcagacatcaatgcagtgaatgaacacgggaatgtgcccctgcactatgcctgtttttggggccaagatcaagtggcagaggacctggtggcaaatggggcccttgtcagcatctgtaacaagtatggagagatgcctgtggacaaagccaaggcacccctgagagagcttctccgagagcgggcagagaagatgggccagaatctcaaccgtattccatacaaggacacattctggaaggggaccacccgcactcggccccgaaatggaaccctgaacaaacactctggcattgacttcaaacagcttaacttcctgacgaagctcaacgagaatcactctggagagctatggaagggccgctggcagggcaatgacattgtcgtgaaggtgctgaaggttcgagactggagtacaaggaagagcagggacttcaatgaagagtgtccccggctcaggattttctcgcatccaaatgtgctcccagtgctaggtgcctgccagtctccacctgctcctcatcctactctcatcacacactggatgccgtatggatccctctacaatgtactacatgaaggcaccaatttcgtcgtggaccagagccaggctgtgaagtttgctttggacatggcaaggggcatggccttcctacacacactagagcccctcatcccacgacatgcactcaatagccgtagtgtaatgattgatgaggacatgactgcccgaattagcatggctgatgtcaagttctctttccaatgtcctggtcgcatgtatgcacctgcctgggtagcccccgaagctctgcagaagaagcctgaagacacaaacagacgctcagcagacatgtggagttttgcagtgcttctgtgggaactggtgacacgggaggtaccctttgctgacctctccaatatggagattggaatgaaggtggcattggaaggccttcggcctaccatcccaccaggtatttcccctcatgtgtgtaagctcatgaagatctgcatgaatgaagaccctgcaaagcgacccaaatttgacatgattgtgcctatccttgagaagatgcaggacaagtaggactggaaggtccttgcctgaactccagaggtgtcgggacatggttgggggaatgcacctccccaaagcagcaggcctctggttgcctcccccgcctccagtcatggtactaccccagccatggggtccatccccttcccccatccctaccactgtggccccaagaggggcgggctcagagctttgtcacttgccacatggtgtctcccaacatgggagggatcagccccgcctgtcacaataaagtttattatgaaaaaaaaaaaaaaaaaaaaaa

SEQ ID NO: 4 - DNA, 1931 bp, Homo sapiens
gggtctagttgcctgctctcggacatccgttcagcagacactacctcttcgtcaccccctgcccaccctgacccgcctttacctcgcgtctagaggacacagccagggatcatcccgcagccccgaactccttcacagacccccactagccggggacgcagctcaggccccctacccccaacacaaacacttctctcctgtagaggataaagcttggggttcatcctccttccctggatcactccacagtcctcaggcttccccaatccaggggactcggcgccgggacgctgctatggacgacattttcactcagtgccgggagggcaacgcagtcgccgttcgcctgtggctggacaacacggagaacgacctcaaccagggggacgatcatggcttctcccccttgcactgggcctgccgagagggccgctctgctgtggttgagatgttgatcatgcggggggcacggatcaatgtaatgaaccgtggggatgacacccccctgcatctggcagccagtcatggacaccgtgatattgtacagaagctattgcagtacaaggcagacatcaatgcagtgaatgaacacgggaatgtgcccctgcactatgcctgtttttggggccaagatcaagtggcagaggacctggtggcaaatggggcccttgtcagcatctgtaacaagtatggagagatgcctgtggacaaagccaaggcacccctgagagagcttctccgagagcgggcagagaagatgggccagaatctcaaccgtattccatacaaggacacattctggaaggggaccacccgcactcggccccgaaatggaaccctgaacaaacactctggcattgacttcaaacagcttaacttcctgacgaagctcaacgagaatcactctggagagctatggaagggccgctggcagggcaatgacattgtcgtgaaggtgctgaaggttcgagactggagtacaaggaagagcagggacttcaatgaagagtgtccccggctcaggattttctcgcatccaaatgtgctcccagtgctaggtgcctgccagtctccacctgctcctcatcctactctcatcacacactggatgccgtatggatccctctacaatgtactacatgaaggcaccaatttcgtcgtggaccagagccaggctgtgaagtttgctttggacatggcaaggggcatggccttcctacacacactagagcccctcatcccacgacatgcactcaatagccgtagtgtaatgattgatgaggacatgactgcccgaattagcatggctgatgtcaagttctctttccaatgtcctggtcgcatgtatgcacctgcctgggtagcccccgaagctctgcagaagaagcctgaagacacaaacagacgctcagcagacatgtggagttttgcagtgcttctgtgggaactggtgacacgggaggtaccctttgctgacctctccaatatggagattggaatgaaggtggcattggaaggccttcggcctaccatcccaccaggtatttcccctcatgtgtgtaagctcatgaagatctgcatgaatgaagaccctgcaaagcgacccaaatttgacatgattgtgcctatccttgagaagatgcaggacaagtaggactggaaggtccttgcctgaactccagaggtgtcgggacatggttgggggaatgcacctccccaaagcagcaggcctctggttgcctcccccgcctccagtcatggtactaccccagccatggggtccatccccttcccccatccctaccactgtggccccaagaggggcgggctcagagctttgtcacttgccacatggtgtctcccaacatgggagggatcagccccgcctgtcacaataaagtttattatgaaaaaaaaaaaaaaaaaaaaaa

SEQ ID NO: 5 - RNA, 25 nt, Artificial Sequence
  Synthesized siRNA molecules designed using the human ILK gene set forth in SEQ ID Nos. 1-4
cagccagucauggacaccgugauau

SEQ ID NO: 6 - RNA, 25 nt, Artificial Sequence
  Synthesized siRNA molecules designed using the human ILK gene set forth in SEQ ID Nos. 1-4
auaucacgguguccaugacuggcug

SEQ ID NO: 7 - RNA, 25 nt, Artificial Sequence
  Synthesized siRNA molecules designed using the human ILK gene set forth in SEQ ID Nos. 1-4
cagucauggacaccgugauauugua

SEQ ID NO: 8 - RNA, 25 nt, Artificial Sequence
  Synthesized siRNA molecules designed using the human ILK gene set forth in SEQ ID Nos. 1-4
uacaauaucacgguguccaugacug

SEQ ID NO: 9 - RNA, 25 nt, Artificial Sequence
  Synthesized siRNA molecules designed using the human ILK gene set forth in SEQ ID Nos. 1-4
cagacaucaaugcagugaaugaaca

SEQ ID NO: 10 - RNA, 25 nt, Artificial Sequence
  Synthesized siRNA molecules designed using the human ILK gene set forth in SEQ ID Nos. 1-4
uguucauucacugcauugaugucug

SEQ ID NO: 11 - RNA, 25 nt, Artificial Sequence
  Synthesized siRNA molecules designed using the human ILK gene set forth in SEQ ID Nos. 1-4
cagagaagaugggccagaaucucaa

SEQ ID NO: 12 - RNA, 25 nt, Artificial Sequence
  Synthesized siRNA molecules designed using the human ILK gene set forth in SEQ ID Nos. 1-4
uugagauucuggcccaucuucucug

SEQ ID NO: 13 - RNA, 25 nt, Artificial Sequence
  Synthesized siRNA molecules designed using the human ILK gene set forth in SEQ ID Nos. 1-4
gccagaaucucaaccguauuccaua

SEQ ID NO: 14 - RNA, 25 nt, Artificial Sequence
  Synthesized siRNA molecules designed using the human ILK gene set forth in SEQ ID Nos. 1-4
uauggaauacgguugagauucuggc

SEQ ID NO: 15 - RNA, 25 nt, Artificial Sequence
  Synthesized siRNA molecules designed using the human ILK gene set forth in SEQ ID Nos. 1-4
gcauugacuucaaacagcuuaacuu

SEQ ID NO: 16 - RNA, 25 nt, Artificial Sequence
  Synthesized siRNA molecules designed using the human ILK gene set forth in SEQ ID Nos. 1-4
aaguuaagcuguuugaagucaaugc

SEQ ID NO: 17 - RNA, 25 nt, Artificial Sequence
  Synthesized siRNA molecules designed using the human ILK gene set forth in SEQ ID Nos. 1-4
gaucccucuacaauguacuacauga

SEQ ID NO: 18 - RNA, 25 nt, Artificial Sequence
  Synthesized siRNA molecules designed using the human ILK gene set forth in SEQ ID Nos. 1-4
ucauguaguacauuguagagggauc

SEQ ID NO: 19 - RNA, 25 nt, Artificial Sequence
  Synthesized siRNA molecules designed using the human ILK gene set forth in SEQ ID Nos. 1-4
gagguacccuuugcugaccucucca

SEQ ID NO: 20 - RNA, 25 nt, Artificial Sequence
  Synthesized siRNA molecules designed using the human ILK gene set forth in SEQ ID Nos. 1-4
uggagaggucagcaaaggguaccuc

SEQ ID NO: 21 - RNA, 25 nt, Artificial Sequence
  Synthesized siRNA molecules designed using the human ILK gene set forth in SEQ ID Nos. 1-4
gauuggaaugaagguggcauuggaa

SEQ ID NO: 22 - RNA, 25 nt, Artificial Sequence
  Synthesized siRNA molecules designed using the human ILK gene set forth in SEQ ID Nos. 1-4
uuccaaugccaccuucauuccaauc

SEQ ID NO: 23 - RNA, 25 nt, Artificial Sequence
  Synthesized siRNA molecules designed using the human ILK gene set forth in SEQ ID Nos. 1-4
ccucauguguguaagcucaugaaga

SEQ ID NO: 24 - RNA, 25 nt, Artificial Sequence
  Synthesized siRNA molecules designed using the human ILK gene set forth in SEQ ID Nos. 1-4
ucuucaugacguuacacacauaagg

SEQ ID NO: 25 - RNA, 25 nt, Artificial Sequence
  Synthesized siRNA molecules designed using the human ILK gene set forth in SEQ ID Nos. 1-4
gcucugcugugguugagauguugau

SEQ ID NO: 26 - RNA, 25 nt, Artificial Sequence
  Synthesized siRNA molecules designed using the human ILK gene set forth in SEQ ID Nos. 1-4
aucaacaucucaaccacagcagagc

SEQ ID NO: 27 - RNA, 25 nt, Artificial Sequence
  Synthesized siRNA molecules designed using the human ILK gene set forth in SEQ ID Nos. 1-4
ucugcugugguugagauguugauca

SEQ ID NO: 28 - RNA, 25 nt, Artificial Sequence
  Synthesized siRNA molecules designed using the human ILK gene set forth in SEQ ID Nos. 1-4
ugaucaacaucucaaccacagcaga

SEQ ID NO: 29 - RNA, 25 nt, Artificial Sequence
  Synthesized siRNA molecules designed using the human ILK gene set forth in SEQ ID Nos. 1-4
ccagucauggacaccgugauauugu

SEQ ID NO: 30 - RNA, 25 nt, Artificial Sequence
  Synthesized siRNA molecules designed using the human ILK gene set forth in SEQ ID Nos. 1-4
acaauaucacgguguccaugacugg

SEQ ID NO: 31 - RNA, 25 nt, Artificial Sequence
  Synthesized siRNA molecules designed using the human ILK gene set forth in SEQ ID Nos. 1-4
caccgugauauuguacagaagcuau

SEQ ID NO: 32 - RNA, 25 nt, Artificial Sequence
  Synthesized siRNA molecules designed using the human ILK gene set forth in SEQ ID Nos. 1-4
auagcuucuguacaauaucacggug

SEQ ID NO: 33 - RNA, 25 nt, Artificial Sequence
  Synthesized siRNA molecules designed using the human ILK gene set forth in SEQ ID Nos. 1-4
accgugauauuguacagaagcuauu

SEQ ID NO: 34 - RNA, 25 nt, Artificial Sequence
  Synthesized siRNA molecules designed using the human ILK gene set forth in SEQ ID Nos. 1-4
aauagcuucuguacaauaucacggu

SEQ ID NO: 35 - RNA, 25 nt, Artificial Sequence
  Synthesized siRNA molecules designed using the human ILK gene set forth in SEQ ID Nos. 1-4
cagaagcuauugcaguacaaggcag

SEQ ID NO: 36 - RNA, 25 nt, Artificial Sequence
  Synthesized siRNA molecules designed using the human ILK gene set forth in SEQ ID Nos. 1-4
cugccuuguacugcaauagcuucug

SEQ ID NO: 37 - RNA, 25 nt, Artificial Sequence
  Synthesized siRNA molecules designed using the human ILK gene set forth in SEQ ID Nos. 1-4
caguacaaggcagacaucaaugcag

SEQ ID NO: 38 - RNA, 25 nt, Artificial Sequence
  Synthesized siRNA molecules designed using the human ILK gene set forth in SEQ ID Nos. 1-4
cugcauugaugucugccuuguacug

SEQ ID NO: 39 - RNA, 25 nt, Artificial Sequence
  Synthesized siRNA molecules designed using the human ILK gene set forth in SEQ ID Nos. 1-4
caaggcagacaucaaugcagugaau

SEQ ID NO: 40 - RNA, 25 nt, Artificial Sequence
  Synthesized siRNA molecules designed using the human ILK gene set forth in SEQ ID Nos. 1-4
auucacugcauugaugucugccuug

SEQ ID NO: 41 - RNA, 25 nt, Artificial Sequence
  Synthesized siRNA molecules designed using the human ILK gene set forth in SEQ ID Nos. 1-4
ucaaugcagugaaugaacacgggaa

SEQ ID NO: 42 - RNA, 25 nt, Artificial Sequence
  Synthesized siRNA molecules designed using the human ILK gene set forth in SEQ ID Nos. 1-4
uucccguguucauucacugcauuga

SEQ ID NO: 43 - RNA, 25 nt, Artificial Sequence
  Synthesized siRNA molecules designed using the human ILK gene set forth in SEQ ID Nos. 1-4
cccuugucagcaucuguaacaagua

SEQ ID NO: 44 - RNA, 25 nt, Artificial Sequence
  Synthesized siRNA molecules designed using the human ILK gene set forth in SEQ ID Nos. 1-4
uacuuguuacagaugcugacaaggg

SEQ ID NO: 45 - RNA, 25 nt, Artificial Sequence
  Synthesized siRNA molecules designed using the human ILK gene set forth in SEQ ID Nos. 1-4
caaacacucuggcauugacuucaaa

SEQ ID NO: 46 - RNA, 25 nt, Artificial Sequence
  Synthesized siRNA molecules designed using the human ILK gene set forth in SEQ ID Nos. 1-4
uuugaagucaaugccagaguguuug

SEQ ID NO: 47 - RNA, 25 nt, Artificial Sequence
  Synthesized siRNA molecules designed using the human ILK gene set forth in SEQ ID Nos. 1-4
ucaaacagcuuaacuuccugacgaa

SEQ ID NO: 48 - RNA, 25 nt, Artificial Sequence
  Synthesized siRNA molecules designed using the human ILK gene set forth in SEQ ID Nos. 1-4
uucgucaggaaguuaagcuguuuga

SEQ ID NO: 49 - RNA, 25 nt, Artificial Sequence
  Synthesized siRNA molecules designed using the human ILK gene set forth in SEQ ID Nos. 1-4
gaagcucaacgagaaucacucugga

SEQ ID NO: 50 - RNA, 25 nt, Artificial Sequence
  Synthesized siRNA molecules designed using the human ILK gene set forth in SEQ ID Nos. 1-4
uccagagugauucucguugagcuuc

SEQ ID NO: 51 - RNA, 25 nt, Artificial Sequence
  Synthesized siRNA molecules designed using the human ILK gene set forth in SEQ ID Nos. 1-4
caacgagaaucacucuggagagcua

SEQ ID NO: 52 - RNA, 25 nt, Artificial Sequence
  Synthesized siRNA molecules designed using the human ILK gene set forth in SEQ ID Nos. 1-4
uagcucuccagagugauucucguug

SEQ ID NO: 53 - RNA, 25 nt, Artificial Sequence
  Synthesized siRNA molecules designed using the human ILK gene set forth in SEQ ID Nos. 1-4
gagaaucacucuggagagcuaugga

SEQ ID NO: 54 - RNA, 25 nt, Artificial Sequence
  Synthesized siRNA molecules designed using the human ILK gene set forth in SEQ ID Nos. 1-4
uccauagcucuccagagugauucuc

SEQ ID NO: 55 - RNA, 25 nt, Artificial Sequence
  Synthesized siRNA molecules designed using the human ILK gene set forth in SEQ ID Nos. 1-4
agaaucacucuggagagcuauggaa

SEQ ID NO: 56 - RNA, 25 nt, Artificial Sequence
  Synthesized siRNA molecules designed using the human ILK gene set forth in SEQ ID Nos. 1-4
uuccauagcucuccagagugauucu

SEQ ID NO: 57 - RNA, 25 nt, Artificial Sequence
  Synthesized siRNA molecules designed using the human ILK gene set forth in SEQ ID Nos. 1-4
ggcagggcaaugacauugucgugaa

SEQ ID NO: 58 - RNA, 25 nt, Artificial Sequence
  Synthesized siRNA molecules designed using the human ILK gene set forth in SEQ ID Nos. 1-4
uucacgacaaugucauugcccugcc

SEQ ID NO: 59 - RNA, 25 nt, Artificial Sequence
  Synthesized siRNA molecules designed using the human ILK gene set forth in SEQ ID Nos. 1-4
gcaaugacauugucgugaaggugcu

SEQ ID NO: 60 - RNA, 25 nt, Artificial Sequence
  Synthesized siRNA molecules designed using the human ILK gene set forth in SEQ ID Nos. 1-4
agcaccuucacgacaaugucauugc

SEQ ID NO: 61 - RNA, 25 nt, Artificial Sequence
  Synthesized siRNA molecules designed using the human ILK gene set forth in SEQ ID Nos. 1-4
ggaagagcagggacuucaaugaaga

SEQ ID NO: 62 - RNA, 25 nt, Artificial Sequence
  Synthesized siRNA molecules designed using the human ILK gene set forth in SEQ ID Nos. 1-4
ucuucauugaagucccugcucuucc

SEQ ID NO: 63 - RNA, 25 nt, Artificial Sequence
  Synthesized siRNA molecules designed using the human ILK gene set forth in SEQ ID Nos. 1-4
caccugcuccucauccuacucucau

SEQ ID NO: 64 - RNA, 25 nt, Artificial Sequence
  Synthesized siRNA molecules designed using the human ILK gene set forth in SEQ ID Nos. 1-4
augagaguaggaugaggagcaggug

SEQ ID NO: 65 - RNA, 25 nt, Artificial Sequence
  Synthesized siRNA molecules designed using the human ILK gene set forth in SEQ ID Nos. 1-4
uggaugccguauggaucccucuaca

SEQ ID NO: 66 - RNA, 25 nt, Artificial Sequence
  Synthesized siRNA molecules designed using the human ILK gene set forth in SEQ ID Nos. 1-4
uguagagggauccauacggcaucca

SEQ ID NO: 67 - RNA, 25 nt, Artificial Sequence
  Synthesized siRNA molecules designed using the human ILK gene set forth in SEQ ID Nos. 1-4
cagagccaggcugugaaguuugcuu

SEQ ID NO: 68 - RNA, 25 nt, Artificial Sequence
  Synthesized siRNA molecules designed using the human ILK gene set forth in SEQ ID Nos. 1-4
aagcaaacuucacagccuggcucug

SEQ ID NO: 69 - RNA, 25 nt, Artificial Sequence
  Synthesized siRNA molecules designed using the human ILK gene set forth in SEQ ID Nos. 1-4
gccaggcugugaaguuugcuuugga

SEQ ID NO: 70 - RNA, 25 nt, Artificial Sequence
  Synthesized siRNA molecules designed using the human ILK gene set forth in SEQ ID Nos. 1-4
uccaaagcaaacuucacagccuggc

SEQ ID NO: 71 - RNA, 25 nt, Artificial Sequence
  Synthesized siRNA molecules designed using the human ILK gene set forth in SEQ ID Nos. 1-4
ccucaucccacgacaugcacucaau

SEQ ID NO: 72 - RNA, 25 nt, Artificial Sequence
  Synthesized siRNA molecules designed using the human ILK gene set forth in SEQ ID Nos. 1-4
auugagugcaugucgugggaugagg

SEQ ID NO: 73 - RNA, 25 nt, Artificial Sequence
  Synthesized siRNA molecules designed using the human ILK gene set forth in SEQ ID Nos. 1-4
ucaauagccguaguguaaugauuga

SEQ ID NO: 74 - RNA, 25 nt, Artificial Sequence
  Synthesized siRNA molecules designed using the human ILK gene set forth in SEQ ID Nos. 1-4
ucaaucauuacacuacggcuauuga

SEQ ID NO: 75 - RNA, 25 nt, Artificial Sequence
  Synthesized siRNA molecules designed using the human ILK gene set forth in SEQ ID Nos. 1-4
ugaugaggacaugacugcccgaauu

SEQ ID NO: 76 - RNA, 25 nt, Artificial Sequence
  Synthesized siRNA molecules designed using the human ILK gene set forth in SEQ ID Nos. 1-4
aauucgggcagucauguccucauca

SEQ ID NO: 77 - RNA, 25 nt, Artificial Sequence
  Synthesized siRNA molecules designed using the human ILK gene set forth in SEQ ID Nos. 1-4
gaggacaugacugcccgaauuagca

SEQ ID NO: 78 - RNA, 25 nt, Artificial Sequence
  Synthesized siRNA molecules designed using the human ILK gene set forth in SEQ ID Nos. 1-4
ugcuaauucgggcagucauguccuc

SEQ ID NO: 79 - RNA, 25 nt, Artificial Sequence
  Synthesized siRNA molecules designed using the human ILK gene set forth in SEQ ID Nos. 1-4
cccgaauuagcauggcugaugucaa

SEQ ID NO: 80 - RNA, 25 nt, Artificial Sequence
  Synthesized siRNA molecules designed using the human ILK gene set forth in SEQ ID Nos. 1-4
uugacaucagccaugcuaauucggg

SEQ ID NO: 81 - RNA, 25 nt, Artificial Sequence
  Synthesized siRNA molecules designed using the human ILK gene set forth in SEQ ID Nos. 1-4
agcauggcugaugucaaguucucuu

SEQ ID NO: 82 - RNA, 25 nt, Artificial Sequence
  Synthesized siRNA molecules designed using the human ILK gene set forth in SEQ ID Nos. 1-4
aagagaacuugacaucagccaugcu

SEQ ID NO: 83 - RNA, 25 nt, Artificial Sequence
  Synthesized siRNA molecules designed using the human ILK gene set forth in SEQ ID Nos. 1-4
uggcugaugucaaguucucuuucca

SEQ ID NO: 84 - RNA, 25 nt, Artificial Sequence
  Synthesized siRNA molecules designed using the human ILK gene set forth in SEQ ID Nos. 1-4
uggaaagagaacuugacaucagcca

SEQ ID NO: 85 - RNA, 25 nt, Artificial Sequence
  Synthesized siRNA molecules designed using the human ILK gene set forth in SEQ ID Nos. 1-4
ggcugaugucaaguucucuuuccaa

SEQ ID NO: 86 - RNA, 25 nt, Artificial Sequence
  Synthesized siRNA molecules designed using the human ILK gene set forth in SEQ ID Nos. 1-4
uuggaaagagaacuugacaucagcc

SEQ ID NO: 87 - RNA, 25 nt, Artificial Sequence
  Synthesized siRNA molecules designed using the human ILK gene set forth in SEQ ID Nos. 1-4
cgaagcucugcagaagaagccugaa

SEQ ID NO: 88 - RNA, 25 nt, Artificial Sequence
  Synthesized siRNA molecules designed using the human ILK gene set forth in SEQ ID Nos. 1-4
uucaggcuucuucugcagagcuucg

SEQ ID NO: 89 - RNA, 25 nt, Artificial Sequence
  Synthesized siRNA molecules designed using the human ILK gene set forth in SEQ ID Nos. 1-4
cagaagaagccugaagacacaaaca

SEQ ID NO: 90 - RNA, 25 nt, Artificial Sequence
  Synthesized siRNA molecules designed using the human ILK gene set forth in SEQ ID Nos. 1-4
uguuugugucuucaggcuucuucug

SEQ ID NO: 91 - RNA, 25 nt, Artificial Sequence
  Synthesized siRNA molecules designed using the human ILK gene set forth in SEQ ID Nos. 1-4
accucuccaauauggagauuggaau

SEQ ID NO: 92 - RNA, 25 nt, Artificial Sequence
  Synthesized siRNA molecules designed using the human ILK gene set forth in SEQ ID Nos. 1-4
auuccaaucuccauauuggagaggu

SEQ ID NO: 93 - RNA, 25 nt, Artificial Sequence
  Synthesized siRNA molecules designed using the human ILK gene set forth in SEQ ID Nos. 1-4
ucaugaagaucugcaugaaugaaga

SEQ ID NO: 94 - RNA, 25 nt, Artificial Sequence
  Synthesized siRNA molecules designed using the human ILK gene set forth in SEQ ID Nos. 1-4
ucuucauucaugcagaucuucauga

SEQ ID NO: 95 - RNA, 25 nt, Artificial Sequence
  Synthesized siRNA molecules designed using the human ILK gene set forth in SEQ ID Nos. 1-4
ucugcaugaaugaagacccugcaaa

SEQ ID NO: 96 - RNA, 25 nt, Artificial Sequence
  Synthesized siRNA molecules designed using the human ILK gene set forth in SEQ ID Nos. 1-4
uuugcagggucuucauucaugcaga

SEQ ID NO: 97 - RNA, 25 nt, Artificial Sequence
  Synthesized siRNA molecules designed using the human ILK gene set forth in SEQ ID Nos. 1-4
gaagacccugcaaagcgacccaaau

SEQ ID NO: 98 - RNA, 25 nt, Artificial Sequence
  Synthesized siRNA molecules designed using the human ILK gene set forth in SEQ ID Nos. 1-4
auuugggucgcuuugcagggucuuc

SEQ ID NO: 99 - RNA, 25 nt, Artificial Sequence
  Synthesized siRNA molecules designed using the human ILK gene set forth in SEQ ID Nos. 1-4
aagacccugcaaagcgacccaaauu

SEQ ID NO: 100 - RNA, 25 nt, Artificial Sequence
  Synthesized siRNA molecules designed using the human ILK gene set forth in SEQ ID Nos. 1-4
aauuugggucgcuuugcagggucuu

SEQ ID NO: 101 - RNA, 25 nt, Artificial Sequence
  Synthesized siRNA molecules designed using the human ILK gene set forth in SEQ ID Nos. 1-4
agacccugcaaagcgacccaaauuu

SEQ ID NO: 102 - RNA, 25 nt, Artificial Sequence
  Synthesized siRNA molecules designed using the human ILK gene set forth in SEQ ID Nos. 1-4
aaauuugggucgcuuugcagggucu

SEQ ID NO: 103 - RNA, 25 nt, Artificial Sequence
  Synthesized siRNA molecules designed using the human ILK gene set forth in SEQ ID Nos. 1-4
caaagcgacccaaauuugacaugau

SEQ ID NO: 104 - RNA, 25 nt, Artificial Sequence
  Synthesized siRNA molecules designed using the human ILK gene set forth in SEQ ID Nos. 1-4
aucaugucaaauuugggucgcuuug

SEQ ID NO: 105 - RNA, 25 nt, Artificial Sequence
  Synthesized siRNA molecules designed using the human ILK gene set forth in SEQ ID Nos. 1-4
aaagcgacccaaauuugacaugauu

SEQ ID NO: 106 - RNA, 25 nt, Artificial Sequence
  Synthesized siRNA molecules designed using the human ILK gene set forth in SEQ ID Nos. 1-4
aucaugucaaauuugggucgcuuug

SEQ ID NO: 107 - RNA, 25 nt, Artificial Sequence
  Synthesized siRNA molecules designed using the human ILK gene set forth in SEQ ID Nos. 1-4
aggacaaguaggacuggaagguccu

SEQ ID NO: 108 - RNA, 25 nt, Artificial Sequence
  Synthesized siRNA molecules designed using the human ILK gene set forth in SEQ ID Nos. 1-4
aggaccuuccaguccuacuuguccu

SEQ ID NO: 109 - RNA, 25 nt, Artificial Sequence
  Synthesized siRNA molecules designed using the human ILK gene set forth in SEQ ID Nos. 1-4
uaggacuggaagguccuugccugaa

SEQ ID NO: 110 - RNA, 25 nt, Artificial Sequence
  Synthesized siRNA molecules designed using the human ILK gene set forth in SEQ ID Nos. 1-4
uucaggcaaggaccuuccaguccua

SEQ ID NO: 111 - RNA, 25 nt, Artificial Sequence
  Synthesized siRNA molecules designed using the human ILK gene set forth in SEQ ID Nos. 1-4
gcucagagcuuugucacuugccaca

SEQ ID NO: 112 - RNA, 25 nt, Artificial Sequence
  Synthesized siRNA molecules designed using the human ILK gene set forth in SEQ ID Nos. 1-4
uguggcaagugacaaagcucugagc

SEQ ID NO: 113 - RNA, 25 nt, Artificial Sequence
  Synthesized siRNA molecules designed using the human ILK gene set forth in SEQ ID Nos. 1-4
cacauggugucuuccaacaugggag

SEQ ID NO: 114 - RNA, 25 nt, Artificial Sequence
  Synthesized siRNA molecules designed using the human ILK gene set forth in SEQ ID Nos. 1-4
cucccauguuggaagacaccaugug

SEQ ID NO: 115 - RNA, 25 nt, Artificial Sequence
  Synthesized siRNA molecules designed using the human ILK gene set forth in SEQ ID Nos. 1-4
cccgccugucacaauaaaguuuauu

SEQ ID NO: 116 - RNA, 25 nt, Artificial Sequence
  Synthesized siRNA molecules designed using the human ILK gene set forth in SEQ ID Nos. 1-4
aauaaacuuuauugugacaggcggg

SEQ ID NO: 117 - RNA, 25 nt, Artificial Sequence
  Synthesized siRNA molecules designed using the human ILK gene set forth in SEQ ID Nos. 1-4
ccgccugucacaauaaaguuuauua

SEQ ID NO: 118 - RNA, 25 nt, Artificial Sequence
  Synthesized siRNA molecules designed using the human ILK gene set forth in SEQ ID Nos. 1-4
uaauaaacuuuauugugacaggcgg

SEQ ID NO: 119 - protein, 452 aa, Homo sapiens
MDDIFTQCREGNAVAVRLWLDNTENDLNQGDDHGFSPLHWACREGRSAVVEMLIMRGARINVMNRGDDTPLHLAASHGHRDIVQKLLQYKADINAVNEHGNVPLHYACFWGQDQVAEDLVANGALVSICNKYGEMPVDKAKAPLRELLRERAEKMGQNLNRIPYKDTFWKGTTRTRPRNGTLNKHSGIDFKQLNFLTKLNENHSGELWKGRWQGNDIVVKVLKVRDWSTRKSRDFNEECPRLRIFSHPNVLPVLGACQSPPAPHPTLITHWMPYGSLYNVLHEGTNFVVDQSQAVKFALDMARGMAFLHTLEPLIPRHALNSRSVMIDEDMTARISMADVKFSFQCPGRMYAPAWVAPEALQKKPEDTNRRSADMWSFAVLLWELVTREVPFADLSNMEIGMKVALEGLRPTIPPGISPHVCKLMKICMNEDPAKRPKFDMIVPILEKMQDK

SEQ ID NO: 120 - protein, 452 aa, Homo sapiens; identical to SEQ ID NO: 119

SEQ ID NO: 121 - protein, 452 aa, Homo sapiens; identical to SEQ ID NO: 119

SEQ ID NO: 122 - protein, 452 aa, Homo sapiens; identical to SEQ ID NO: 119